SEQUENCE LISTING (243 sequences)

SEQ ID NO: 1 - protein, 388 aa, Artificial Sequence
  Amino acid sequence of modified parkin
QEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 2 - protein, 12 aa, Artificial Sequence
  Amino acid Sequence of aMTD1
AAALAPVVLALP

SEQ ID NO: 3 - protein, 12 aa, Artificial Sequence
  Amino acid Sequence of aMTD2
AAAVPLLAVVVP

SEQ ID NO: 4 - protein, 12 aa, Artificial Sequence
  Amino acid Sequence of aMTD3
AALLVPAAVLAP

SEQ ID NO: 5 - protein, 12 aa, Artificial Sequence
  Amino acid Sequence of aMTD4
ALALLPVAALAP

SEQ ID NO: 6 - protein, 12 aa, Artificial Sequence
  Amino acid Sequence of aMTD5
AAALLPVALVAP

SEQ ID NO: 7 - protein, 12 aa, Artificial Sequence
  Amino acid Sequence of aMTD11
VVALAPALAALP

SEQ ID NO: 8 - protein, 12 aa, Artificial Sequence
  Amino acid Sequence of aMTD12
LLAAVPAVLLAP

SEQ ID NO: 9 - protein, 12 aa, Artificial Sequence
  Amino acid Sequence of aMTD13
AAALVPVVALLP

SEQ ID NO: 10 - protein, 12 aa, Artificial Sequence
  Amino acid Sequence of aMTD21
AVALLPALLAVP

SEQ ID NO: 11 - protein, 12 aa, Artificial Sequence
  Amino acid Sequence of aMTD22
AVVLVPVLAAAP

SEQ ID NO: 12 - protein, 12 aa, Artificial Sequence
  Amino acid Sequence of aMTD23
VVLVLPAAAAVP

SEQ ID NO: 13 - protein, 12 aa, Artificial Sequence
  Amino acid Sequence of aMTD24
IALAAPALIVAP

SEQ ID NO: 14 - protein, 12 aa, Artificial Sequence
  Amino acid Sequence of aMTD25
IVAVAPALVALP

SEQ ID NO: 15 - protein, 12 aa, Artificial Sequence
  Amino acid Sequence of aMTD42
VAALPVVAVVAP

SEQ ID NO: 16 - protein, 12 aa, Artificial Sequence
  Amino acid Sequence of aMTD43
LLAAPLVVAAVP

SEQ ID NO: 17 - protein, 12 aa, Artificial Sequence
  Amino acid Sequence of aMTD44
ALAVPVALLVAP

SEQ ID NO: 18 - protein, 12 aa, Artificial Sequence
  Amino acid Sequence of aMTD61
VAALPVLLAALP

SEQ ID NO: 19 - protein, 12 aa, Artificial Sequence
  Amino acid Sequence of aMTD62
VALLAPVALAVP

SEQ ID NO: 20 - protein, 12 aa, Artificial Sequence
  Amino acid Sequence of aMTD63
AALLVPALVAVP

SEQ ID NO: 21 - protein, 12 aa, Artificial Sequence
  Amino acid Sequence of aMTD64
AIVALPVAVLAP

SEQ ID NO: 22 - protein, 12 aa, Artificial Sequence
  Amino acid Sequence of aMTD65
IAIVAPVVALAP

SEQ ID NO: 23 - protein, 12 aa, Artificial Sequence
  Amino acid Sequence of aMTD81
AALLPALAALLP

SEQ ID NO: 24 - protein, 12 aa, Artificial Sequence
  Amino acid Sequence of aMTD82
AVVLAPVAAVLP

SEQ ID NO: 25 - protein, 12 aa, Artificial Sequence
  Amino acid Sequence of aMTD83
LAVAAPLALALP

SEQ ID NO: 26 - protein, 12 aa, Artificial Sequence
  Amino acid Sequence of aMTD84
AAVAAPLLLALP

SEQ ID NO: 27 - protein, 12 aa, Artificial Sequence
  Amino acid Sequence of aMTD85
LLVLPAAALAAP

SEQ ID NO: 28 - protein, 12 aa, Artificial Sequence
  Amino acid Sequence of aMTD101
LVALAPVAAVLP

SEQ ID NO: 29 - protein, 12 aa, Artificial Sequence
  Amino acid Sequence of aMTD102
LALAPAALALLP

SEQ ID NO: 30 - protein, 12 aa, Artificial Sequence
  Amino acid Sequence of aMTD103
ALIAAPILALAP

SEQ ID NO: 31 - protein, 12 aa, Artificial Sequence
  Amino acid Sequence of aMTD104
AVVAAPLVLALP

SEQ ID NO: 32 - protein, 12 aa, Artificial Sequence
  Amino acid Sequence of aMTD105
LLALAPAALLAP

SEQ ID NO: 33 - protein, 12 aa, Artificial Sequence
  Amino acid Sequence of aMTD121
AIVALPALALAP

SEQ ID NO: 34 - protein, 12 aa, Artificial Sequence
  Amino acid Sequence of aMTD123
AAIIVPAALLAP

SEQ ID NO: 35 - protein, 12 aa, Artificial Sequence
  Amino acid Sequence of aMTD124
IAVALPALIAAP

SEQ ID NO: 36 - protein, 12 aa, Artificial Sequence
  Amino acid Sequence of aMTD141
AVIVLPALAVAP

SEQ ID NO: 37 - protein, 12 aa, Artificial Sequence
  Amino acid Sequence of aMTD143
AVLAVPAVLVAP

SEQ ID NO: 38 - protein, 12 aa, Artificial Sequence
  Amino acid Sequence of aMTD144
VLAIVPAVALAP

SEQ ID NO: 39 - protein, 12 aa, Artificial Sequence
  Amino acid Sequence of aMTD145
LLAVVPAVALAP

SEQ ID NO: 40 - protein, 12 aa, Artificial Sequence
  Amino acid Sequence of aMTD161
AVIALPALIAAP

SEQ ID NO: 41 - protein, 12 aa, Artificial Sequence
  Amino acid Sequence of aMTD162
AVVALPAALIVP

SEQ ID NO: 42 - protein, 12 aa, Artificial Sequence
  Amino acid Sequence of aMTD163
LALVLPAALAAP

SEQ ID NO: 43 - protein, 12 aa, Artificial Sequence
  Amino acid Sequence of aMTD164
LAAVLPALLAAP

SEQ ID NO: 44 - protein, 12 aa, Artificial Sequence
  Amino acid Sequence of aMTD165
ALAVPVALAIVP

SEQ ID NO: 45 - protein, 12 aa, Artificial Sequence
  Amino acid Sequence of aMTD182
ALIAPVVALVAP

SEQ ID NO: 46 - protein, 12 aa, Artificial Sequence
  Amino acid Sequence of aMTD183
LLAAPVVIALAP

SEQ ID NO: 47 - protein, 12 aa, Artificial Sequence
  Amino acid Sequence of aMTD184
LAAIVPAIIAVP

SEQ ID NO: 48 - protein, 12 aa, Artificial Sequence
  Amino acid Sequence of aMTD185
AALVLPLIIAAP

SEQ ID NO: 49 - protein, 12 aa, Artificial Sequence
  Amino acid Sequence of aMTD201
LALAVPALAALP

SEQ ID NO: 50 - protein, 12 aa, Artificial Sequence
  Amino acid Sequence of aMTD204
LIAALPAVAALP

SEQ ID NO: 51 - protein, 12 aa, Artificial Sequence
  Amino acid Sequence of aMTD205
ALALVPAIAALP

SEQ ID NO: 52 - protein, 12 aa, Artificial Sequence
  Amino acid Sequence of aMTD221
AAILAPIVALAP

SEQ ID NO: 53 - protein, 12 aa, Artificial Sequence
  Amino acid Sequence of aMTD222
ALLIAPAAVIAP

SEQ ID NO: 54 - protein, 12 aa, Artificial Sequence
  Amino acid Sequence of aMTD223
AILAVPIAVVAP

SEQ ID NO: 55 - protein, 12 aa, Artificial Sequence
  Amino acid Sequence of aMTD224
ILAAVPIALAAP

SEQ ID NO: 56 - protein, 12 aa, Artificial Sequence
  Amino acid Sequence of aMTD225
VAALLPAAAVLP

SEQ ID NO: 57 - protein, 12 aa, Artificial Sequence
  Amino acid Sequence of aMTD241
AAAVVPVLLVAP

SEQ ID NO: 58 - protein, 12 aa, Artificial Sequence
  Amino acid Sequence of aMTD242
AALLVPALVAAP

SEQ ID NO: 59 - protein, 12 aa, Artificial Sequence
  Amino acid Sequence of aMTD243
AAVLLPVALAAP

SEQ ID NO: 60 - protein, 12 aa, Artificial Sequence
  Amino acid Sequence of aMTD245
AAALAPVLALVP

SEQ ID NO: 61 - protein, 12 aa, Artificial Sequence
  Amino acid Sequence of aMTD261
LVLVPLLAAAAP

SEQ ID NO: 62 - protein, 12 aa, Artificial Sequence
  Amino acid Sequence of aMTD262
ALIAVPAIIVAP

SEQ ID NO: 63 - protein, 12 aa, Artificial Sequence
  Amino acid Sequence of aMTD263
ALAVIPAAAILP

SEQ ID NO: 64 - protein, 12 aa, Artificial Sequence
  Amino acid Sequence of aMTD264
LAAAPVVIVIAP

SEQ ID NO: 65 - protein, 12 aa, Artificial Sequence
  Amino acid Sequence of aMTD265
VLAIAPLLAAVP

SEQ ID NO: 66 - protein, 12 aa, Artificial Sequence
  Amino acid Sequence of aMTD281
ALIVLPAAVAVP

SEQ ID NO: 67 - protein, 12 aa, Artificial Sequence
  Amino acid Sequence of aMTD282
VLAVAPALIVAP

SEQ ID NO: 68 - protein, 12 aa, Artificial Sequence
  Amino acid Sequence of aMTD283
AALLAPALIVAP

SEQ ID NO: 69 - protein, 12 aa, Artificial Sequence
  Amino acid Sequence of aMTD284
ALIAPAVALIVP

SEQ ID NO: 70 - protein, 12 aa, Artificial Sequence
  Amino acid Sequence of aMTD285
AIVLLPAAVVAP

SEQ ID NO: 71 - protein, 12 aa, Artificial Sequence
  Amino acid Sequence of aMTD301
VIAAPVLAVLAP

SEQ ID NO: 72 - protein, 12 aa, Artificial Sequence
  Amino acid Sequence of aMTD302
LALAPALALLAP

SEQ ID NO: 73 - protein, 12 aa, Artificial Sequence
  Amino acid Sequence of aMTD304
AIILAPIAAIAP

SEQ ID NO: 74 - protein, 12 aa, Artificial Sequence
  Amino acid Sequence of aMTD305
IALAAPILLAAP

SEQ ID NO: 75 - protein, 12 aa, Artificial Sequence
  Amino acid Sequence of aMTD321
IVAVALPALAVP

SEQ ID NO: 76 - protein, 12 aa, Artificial Sequence
  Amino acid Sequence of aMTD322
VVAIVLPALAAP

SEQ ID NO: 77 - protein, 12 aa, Artificial Sequence
  Amino acid Sequence of aMTD323
IVAVALPVALAP

SEQ ID NO: 78 - protein, 12 aa, Artificial Sequence
  Amino acid Sequence of aMTD324
IVAVALPAALVP

SEQ ID NO: 79 - protein, 12 aa, Artificial Sequence
  Amino acid Sequence of aMTD325
IVAVALPAVALP

SEQ ID NO: 80 - protein, 12 aa, Artificial Sequence
  Amino acid Sequence of aMTD341
IVAVALPAVLAP

SEQ ID NO: 81 - protein, 12 aa, Artificial Sequence
  Amino acid Sequence of aMTD342
VIVALAPAVLAP

SEQ ID NO: 82 - protein, 12 aa, Artificial Sequence
  Amino acid Sequence of aMTD343
IVAVALPALVAP

SEQ ID NO: 83 - protein, 12 aa, Artificial Sequence
  Amino acid Sequence of aMTD345
ALLIVAPVAVAP

SEQ ID NO: 84 - protein, 12 aa, Artificial Sequence
  Amino acid Sequence of aMTD361
AVVIVAPAVIAP

SEQ ID NO: 85 - protein, 12 aa, Artificial Sequence
  Amino acid Sequence of aMTD363
AVLAVAPALIVP

SEQ ID NO: 86 - protein, 12 aa, Artificial Sequence
  Amino acid Sequence of aMTD364
LVAAVAPALIVP

SEQ ID NO: 87 - protein, 12 aa, Artificial Sequence
  Amino acid Sequence of aMTD365
AVIVVAPALLAP

SEQ ID NO: 88 - protein, 12 aa, Artificial Sequence
  Amino acid Sequence of aMTD381
VVAIVLPAVAAP

SEQ ID NO: 89 - protein, 12 aa, Artificial Sequence
  Amino acid Sequence of aMTD382
AAALVIPAILAP

SEQ ID NO: 90 - protein, 12 aa, Artificial Sequence
  Amino acid Sequence of aMTD383
VIVALAPALLAP

SEQ ID NO: 91 - protein, 12 aa, Artificial Sequence
  Amino acid Sequence of aMTD384
VIVAIAPALLAP

SEQ ID NO: 92 - protein, 12 aa, Artificial Sequence
  Amino acid Sequence of aMTD385
IVAIAVPALVAP

SEQ ID NO: 93 - protein, 12 aa, Artificial Sequence
  Amino acid Sequence of aMTD401
AALAVIPAAILP

SEQ ID NO: 94 - protein, 12 aa, Artificial Sequence
  Amino acid Sequence of aMTD402
ALAAVIPAAILP

SEQ ID NO: 95 - protein, 12 aa, Artificial Sequence
  Amino acid Sequence of aMTD403
AAALVIPAAILP

SEQ ID NO: 96 - protein, 12 aa, Artificial Sequence
  Amino acid Sequence of aMTD404
LAAAVIPAAILP

SEQ ID NO: 97 - protein, 12 aa, Artificial Sequence
  Amino acid Sequence of aMTD405
LAAAVIPVAILP

SEQ ID NO: 98 - protein, 12 aa, Artificial Sequence
  Amino acid Sequence of aMTD421
AAILAAPLIAVP

SEQ ID NO: 99 - protein, 12 aa, Artificial Sequence
  Amino acid Sequence of aMTD422
VVAILAPLLAAP

SEQ ID NO: 100 - protein, 12 aa, Artificial Sequence
  Amino acid Sequence of aMTD424
AVVVAAPVLALP

SEQ ID NO: 101 - protein, 12 aa, Artificial Sequence
  Amino acid Sequence of aMTD425
AVVAIAPVLALP

SEQ ID NO: 102 - protein, 12 aa, Artificial Sequence
  Amino acid Sequence of aMTD442
ALAALVPAVLVP

SEQ ID NO: 103 - protein, 12 aa, Artificial Sequence
  Amino acid Sequence of aMTD443
ALAALVPVALVP

SEQ ID NO: 104 - protein, 12 aa, Artificial Sequence
  Amino acid Sequence of aMTD444
LAAALVPVALVP

SEQ ID NO: 105 - protein, 12 aa, Artificial Sequence
  Amino acid Sequence of aMTD445
ALAALVPALVVP

SEQ ID NO: 106 - protein, 12 aa, Artificial Sequence
  Amino acid Sequence of aMTD461
IAAVIVPAVALP

SEQ ID NO: 107 - protein, 12 aa, Artificial Sequence
  Amino acid Sequence of aMTD462
IAAVLVPAVALP

SEQ ID NO: 108 - protein, 12 aa, Artificial Sequence
  Amino acid Sequence of aMTD463
AVAILVPLLAAP

SEQ ID NO: 109 - protein, 12 aa, Artificial Sequence
  Amino acid Sequence of aMTD464
AVVILVPLAAAP

SEQ ID NO: 110 - protein, 12 aa, Artificial Sequence
  Amino acid Sequence of aMTD465
IAAVIVPVAALP

SEQ ID NO: 111 - protein, 12 aa, Artificial Sequence
  Amino acid Sequence of aMTD481
AIAIAIVPVALP

SEQ ID NO: 112 - protein, 12 aa, Artificial Sequence
  Amino acid Sequence of aMTD482
ILAVAAIPVAVP

SEQ ID NO: 113 - protein, 12 aa, Artificial Sequence
  Amino acid Sequence of aMTD483
ILAAAIIPAALP

SEQ ID NO: 114 - protein, 12 aa, Artificial Sequence
  Amino acid Sequence of aMTD484
LAVVLAAPAIVP

SEQ ID NO: 115 - protein, 12 aa, Artificial Sequence
  Amino acid Sequence of aMTD485
AILAAIVPLAVP

SEQ ID NO: 116 - protein, 12 aa, Artificial Sequence
  Amino acid Sequence of aMTD501
VIVALAVPALAP

SEQ ID NO: 117 - protein, 12 aa, Artificial Sequence
  Amino acid Sequence of aMTD502
AIVALAVPVLAP

SEQ ID NO: 118 - protein, 12 aa, Artificial Sequence
  Amino acid Sequence of aMTD503
AAIIIVLPAALP

SEQ ID NO: 119 - protein, 12 aa, Artificial Sequence
  Amino acid Sequence of aMTD504
LIVALAVPALAP

SEQ ID NO: 120 - protein, 12 aa, Artificial Sequence
  Amino acid Sequence of aMTD505
AIIIVIAPAAAP

SEQ ID NO: 121 - protein, 12 aa, Artificial Sequence
  Amino acid Sequence of aMTD521
LAALIVVPAVAP

SEQ ID NO: 122 - protein, 12 aa, Artificial Sequence
  Amino acid Sequence of aMTD522
ALLVIAVPAVAP

SEQ ID NO: 123 - protein, 12 aa, Artificial Sequence
  Amino acid Sequence of aMTD524
AVALIVVPALAP

SEQ ID NO: 124 - protein, 12 aa, Artificial Sequence
  Amino acid Sequence of aMTD525
ALAIVVAPVAVP

SEQ ID NO: 125 - protein, 12 aa, Artificial Sequence
  Amino acid Sequence of aMTD541
LLALIIAPAAAP

SEQ ID NO: 126 - protein, 12 aa, Artificial Sequence
  Amino acid Sequence of aMTD542
ALALIIVPAVAP

SEQ ID NO: 127 - protein, 12 aa, Artificial Sequence
  Amino acid Sequence of aMTD543
LLAALIAPAALP

SEQ ID NO: 128 - protein, 12 aa, Artificial Sequence
  Amino acid Sequence of aMTD544
IVALIVAPAAVP

SEQ ID NO: 129 - protein, 12 aa, Artificial Sequence
  Amino acid Sequence of aMTD545
VVLVLAAPAAVP

SEQ ID NO: 130 - protein, 12 aa, Artificial Sequence
  Amino acid Sequence of aMTD561
AAVAIVLPAVVP

SEQ ID NO: 131 - protein, 12 aa, Artificial Sequence
  Amino acid Sequence of aMTD562
ALIAAIVPALVP

SEQ ID NO: 132 - protein, 12 aa, Artificial Sequence
  Amino acid Sequence of aMTD563
ALAVIVVPALAP

SEQ ID NO: 133 - protein, 12 aa, Artificial Sequence
  Amino acid Sequence of aMTD564
VAIALIVPALAP

SEQ ID NO: 134 - protein, 12 aa, Artificial Sequence
  Amino acid Sequence of aMTD565
VAIVLVAPAVAP

SEQ ID NO: 135 - protein, 12 aa, Artificial Sequence
  Amino acid Sequence of aMTD582
VAVALIVPALAP

SEQ ID NO: 136 - protein, 12 aa, Artificial Sequence
  Amino acid Sequence of aMTD583
AVILALAPIVAP

SEQ ID NO: 137 - protein, 12 aa, Artificial Sequence
  Amino acid Sequence of aMTD585
ALIVAIAPALVP

SEQ ID NO: 138 - protein, 12 aa, Artificial Sequence
  Amino acid Sequence of aMTD601
AAILIAVPIAAP

SEQ ID NO: 139 - protein, 12 aa, Artificial Sequence
  Amino acid Sequence of aMTD602
VIVALAAPVLAP

SEQ ID NO: 140 - protein, 12 aa, Artificial Sequence
  Amino acid Sequence of aMTD603
VLVALAAPVIAP

SEQ ID NO: 141 - protein, 12 aa, Artificial Sequence
  Amino acid Sequence of aMTD604
VALIAVAPAVVP

SEQ ID NO: 142 - protein, 12 aa, Artificial Sequence
  Amino acid Sequence of aMTD605
VIAAVLAPVAVP

SEQ ID NO: 143 - protein, 12 aa, Artificial Sequence
  Amino acid Sequence of aMTD622
ALIVLAAPVAVP

SEQ ID NO: 144 - protein, 12 aa, Artificial Sequence
  Amino acid Sequence of aMTD623
VAAAIALPAIVP

SEQ ID NO: 145 - protein, 12 aa, Artificial Sequence
  Amino acid Sequence of aMTD625
ILAAAAAPLIVP

SEQ ID NO: 146 - protein, 12 aa, Artificial Sequence
  Amino acid Sequence of aMTD643
LALVLAAPAIVP

SEQ ID NO: 147 - protein, 12 aa, Artificial Sequence
  Amino acid Sequence of aMTD645
ALAVVALPAIVP

SEQ ID NO: 148 - protein, 12 aa, Artificial Sequence
  Amino acid Sequence of aMTD661
AAILAPIVAALP

SEQ ID NO: 149 - protein, 12 aa, Artificial Sequence
  Amino acid Sequence of aMTD664
ILIAIAIPAAAP

SEQ ID NO: 150 - protein, 12 aa, Artificial Sequence
  Amino acid Sequence of aMTD665
LAIVLAAPVAVP

SEQ ID NO: 151 - protein, 12 aa, Artificial Sequence
  Amino acid Sequence of aMTD666
AAIAIIAPAIVP

SEQ ID NO: 152 - protein, 12 aa, Artificial Sequence
  Amino acid Sequence of aMTD667
LAVAIVAPALVP

SEQ ID NO: 153 - protein, 12 aa, Artificial Sequence
  Amino acid Sequence of aMTD683
LAIVLAAPAVLP

SEQ ID NO: 154 - protein, 12 aa, Artificial Sequence
  Amino acid Sequence of aMTD684
AAIVLALPAVLP

SEQ ID NO: 155 - protein, 12 aa, Artificial Sequence
  Amino acid Sequence of aMTD685
ALLVAVLPAALP

SEQ ID NO: 156 - protein, 12 aa, Artificial Sequence
  Amino acid Sequence of aMTD686
AALVAVLPVALP

SEQ ID NO: 157 - protein, 12 aa, Artificial Sequence
  Amino acid Sequence of aMTD687
AILAVALPLLAP

SEQ ID NO: 158 - protein, 12 aa, Artificial Sequence
  Amino acid Sequence of aMTD703
IVAVALVPALAP

SEQ ID NO: 159 - protein, 12 aa, Artificial Sequence
  Amino acid Sequence of aMTD705
IVAVALLPALAP

SEQ ID NO: 160 - protein, 12 aa, Artificial Sequence
  Amino acid Sequence of aMTD706
IVAVALLPAVAP

SEQ ID NO: 161 - protein, 12 aa, Artificial Sequence
  Amino acid Sequence of aMTD707
IVALAVLPAVAP

SEQ ID NO: 162 - protein, 12 aa, Artificial Sequence
  Amino acid Sequence of aMTD724
VAVLAVLPALAP

SEQ ID NO: 163 - protein, 12 aa, Artificial Sequence
  Amino acid Sequence of aMTD725
IAVLAVAPAVLP

SEQ ID NO: 164 - protein, 12 aa, Artificial Sequence
  Amino acid Sequence of aMTD726
LAVAIIAPAVAP

SEQ ID NO: 165 - protein, 12 aa, Artificial Sequence
  Amino acid Sequence of aMTD727
VALAIALPAVLP

SEQ ID NO: 166 - protein, 12 aa, Artificial Sequence
  Amino acid Sequence of aMTD743
AIAIALVPVALP

SEQ ID NO: 167 - protein, 12 aa, Artificial Sequence
  Amino acid Sequence of aMTD744
AAVVIVAPVALP

SEQ ID NO: 168 - protein, 12 aa, Artificial Sequence
  Amino acid Sequence of aMTD746
VAIIVVAPALAP

SEQ ID NO: 169 - protein, 12 aa, Artificial Sequence
  Amino acid Sequence of aMTD747
VALLAIAPALAP

SEQ ID NO: 170 - protein, 12 aa, Artificial Sequence
  Amino acid Sequence of aMTD763
VAVLIAVPALAP

SEQ ID NO: 171 - protein, 12 aa, Artificial Sequence
  Amino acid Sequence of aMTD764
AVALAVLPAVVP

SEQ ID NO: 172 - protein, 12 aa, Artificial Sequence
  Amino acid Sequence of aMTD765
AVALAVVPAVLP

SEQ ID NO: 173 - protein, 12 aa, Artificial Sequence
  Amino acid Sequence of aMTD766
IVVIAVAPAVAP

SEQ ID NO: 174 - protein, 12 aa, Artificial Sequence
  Amino acid Sequence of aMTD767
IVVAAVVPALAP

SEQ ID NO: 175 - protein, 12 aa, Artificial Sequence
  Amino acid Sequence of aMTD783
IVALVPAVAIAP

SEQ ID NO: 176 - protein, 12 aa, Artificial Sequence
  Amino acid Sequence of aMTD784
VAALPAVALVVP

SEQ ID NO: 177 - protein, 12 aa, Artificial Sequence
  Amino acid Sequence of aMTD786
LVAIAPLAVLAP

SEQ ID NO: 178 - protein, 12 aa, Artificial Sequence
  Amino acid Sequence of aMTD787
AVALVPVIVAAP

SEQ ID NO: 179 - protein, 12 aa, Artificial Sequence
  Amino acid Sequence of aMTD788
AIAVAIAPVALP

SEQ ID NO: 180 - protein, 12 aa, Artificial Sequence
  Amino acid Sequence of aMTD803
AIALAVPVLALP

SEQ ID NO: 181 - protein, 12 aa, Artificial Sequence
  Amino acid Sequence of aMTD805
LVLIAAAPIALP

SEQ ID NO: 182 - protein, 12 aa, Artificial Sequence
  Amino acid Sequence of aMTD806
LVALAVPAAVLP

SEQ ID NO: 183 - protein, 12 aa, Artificial Sequence
  Amino acid Sequence of aMTD807
AVALAVPALVLP

SEQ ID NO: 184 - protein, 12 aa, Artificial Sequence
  Amino acid Sequence of aMTD808
LVVLAAAPLAVP

SEQ ID NO: 185 - protein, 12 aa, Artificial Sequence
  Amino acid Sequence of aMTD809
LIVLAAPALAAP

SEQ ID NO: 186 - protein, 12 aa, Artificial Sequence
  Amino acid Sequence of aMTD810
VIVLAAPALAAP

SEQ ID NO: 187 - protein, 12 aa, Artificial Sequence
  Amino acid Sequence of aMTD811
AVVLAVPALAVP

SEQ ID NO: 188 - protein, 12 aa, Artificial Sequence
  Amino acid Sequence of aMTD824
LIIVAAAPAVAP

SEQ ID NO: 189 - protein, 12 aa, Artificial Sequence
  Amino acid Sequence of aMTD825
IVAVIVAPAVAP

SEQ ID NO: 190 - protein, 12 aa, Artificial Sequence
  Amino acid Sequence of aMTD826
LVALAAPIIAVP

SEQ ID NO: 191 - protein, 12 aa, Artificial Sequence
  Amino acid Sequence of aMTD827
IAAVLAAPALVP

SEQ ID NO: 192 - protein, 12 aa, Artificial Sequence
  Amino acid Sequence of aMTD828
IALLAAPIIAVP

SEQ ID NO: 193 - protein, 12 aa, Artificial Sequence
  Amino acid Sequence of aMTD829
AALALVAPVIVP

SEQ ID NO: 194 - protein, 12 aa, Artificial Sequence
  Amino acid Sequence of aMTD830
IALVAAPVALVP

SEQ ID NO: 195 - protein, 12 aa, Artificial Sequence
  Amino acid Sequence of aMTD831
IIVAVAPAAIVP

SEQ ID NO: 196 - protein, 12 aa, Artificial Sequence
  Amino acid Sequence of aMTD832
AVAAIVPVIVAP

SEQ ID NO: 197 - protein, 12 aa, Artificial Sequence
  Amino acid Sequence of aMTD843
AVLVLVAPAAAP

SEQ ID NO: 198 - protein, 12 aa, Artificial Sequence
  Amino acid Sequence of aMTD844
VVALLAPLIAAP

SEQ ID NO: 199 - protein, 12 aa, Artificial Sequence
  Amino acid Sequence of aMTD845
AAVVIAPLLAVP

SEQ ID NO: 200 - protein, 12 aa, Artificial Sequence
  Amino acid Sequence of aMTD846
IAVAVAAPLLVP

SEQ ID NO: 201 - protein, 12 aa, Artificial Sequence
  Amino acid Sequence of aMTD847
LVAIVVLPAVAP

SEQ ID NO: 202 - protein, 12 aa, Artificial Sequence
  Amino acid Sequence of aMTD848
AVAIVVLPAVAP

SEQ ID NO: 203 - protein, 12 aa, Artificial Sequence
  Amino acid Sequence of aMTD849
AVILLAPLIAAP

SEQ ID NO: 204 - protein, 12 aa, Artificial Sequence
  Amino acid Sequence of aMTD850
LVIALAAPVALP

SEQ ID NO: 205 - protein, 12 aa, Artificial Sequence
  Amino acid Sequence of aMTD851
VLAVVLPAVALP

SEQ ID NO: 206 - protein, 12 aa, Artificial Sequence
  Amino acid Sequence of aMTD852
VLAVAAPAVLLP

SEQ ID NO: 207 - protein, 12 aa, Artificial Sequence
  Amino acid Sequence of aMTD863
AAVVLLPIIAAP

SEQ ID NO: 208 - protein, 12 aa, Artificial Sequence
  Amino acid Sequence of aMTD864
ALLVIAPAIAVP

SEQ ID NO: 209 - protein, 12 aa, Artificial Sequence
  Amino acid Sequence of aMTD865
AVLVIAVPAIAP

SEQ ID NO: 210 - protein, 12 aa, Artificial Sequence
  Amino acid Sequence of aMTD867
ALLVVIAPLAAP

SEQ ID NO: 211 - protein, 12 aa, Artificial Sequence
  Amino acid Sequence of aMTD868
VLVAAILPAAIP

SEQ ID NO: 212 - protein, 12 aa, Artificial Sequence
  Amino acid Sequence of aMTD870
VLVAAVLPIAAP

SEQ ID NO: 213 - protein, 12 aa, Artificial Sequence
  Amino acid Sequence of aMTD872
VLAAAVLPLVVP

SEQ ID NO: 214 - protein, 12 aa, Artificial Sequence
  Amino acid Sequence of aMTD875
AIAIVVPAVAVP

SEQ ID NO: 215 - protein, 12 aa, Artificial Sequence
  Amino acid Sequence of aMTD877
VAIIAVPAVVAP

SEQ ID NO: 216 - protein, 12 aa, Artificial Sequence
  Amino acid Sequence of aMTD878
IVALVAPAAVVP

SEQ ID NO: 217 - protein, 12 aa, Artificial Sequence
  Amino acid Sequence of aMTD879
AAIVLLPAVVVP

SEQ ID NO: 218 - protein, 12 aa, Artificial Sequence
  Amino acid Sequence of aMTD881
AALIVVPAVAVP

SEQ ID NO: 219 - protein, 12 aa, Artificial Sequence
  Amino acid Sequence of aMTD882
AIALVVPAVAVP

SEQ ID NO: 220 - protein, 12 aa, Artificial Sequence
  Amino acid Sequence of aMTD883
LAIVPAAIAALP

SEQ ID NO: 221 - protein, 12 aa, Artificial Sequence
  Amino acid Sequence of aMTD885
LVAIAPAVAVLP

SEQ ID NO: 222 - protein, 12 aa, Artificial Sequence
  Amino acid Sequence of aMTD887
VLAVAPAVAVLP

SEQ ID NO: 223 - protein, 12 aa, Artificial Sequence
  Amino acid Sequence of aMTD888
ILAVVAIPAAAP

SEQ ID NO: 224 - protein, 12 aa, Artificial Sequence
  Amino acid Sequence of aMTD889
ILVAAAPIAALP

SEQ ID NO: 225 - protein, 12 aa, Artificial Sequence
  Amino acid Sequence of aMTD891
ILAVAAIPAALP

SEQ ID NO: 226 - protein, 12 aa, Artificial Sequence
  Amino acid Sequence of aMTD893
VIAIPAILAAAP

SEQ ID NO: 227 - protein, 12 aa, Artificial Sequence
  Amino acid Sequence of aMTD895
AIIIVVPAIAAP

SEQ ID NO: 228 - protein, 12 aa, Artificial Sequence
  Amino acid Sequence of aMTD896
AILIVVAPIAAP

SEQ ID NO: 229 - protein, 12 aa, Artificial Sequence
  Amino acid Sequence of aMTD897
AVIVPVAIIAAP

SEQ ID NO: 230 - protein, 12 aa, Artificial Sequence
  Amino acid Sequence of aMTD899
AVVIALPAVVAP

SEQ ID NO: 231 - protein, 12 aa, Artificial Sequence
  Amino acid Sequence of aMTD900
ALVAVIAPVVAP

SEQ ID NO: 232 - protein, 12 aa, Artificial Sequence
  Amino acid Sequence of aMTD901
ALVAVLPAVAVP

SEQ ID NO: 233 - protein, 12 aa, Artificial Sequence
  Amino acid Sequence of aMTD902
ALVAPLLAVAVP

SEQ ID NO: 234 - protein, 12 aa, Artificial Sequence
  Amino acid Sequence of aMTD904
AVLAVVAPVVAP

SEQ ID NO: 235 - protein, 12 aa, Artificial Sequence
  Amino acid Sequence of aMTD905
AVIAVAPLVVAP

SEQ ID NO: 236 - protein, 12 aa, Artificial Sequence
  Amino acid Sequence of aMTD906
AVIALAPVVVAP

SEQ ID NO: 237 - protein, 12 aa, Artificial Sequence
  Amino acid Sequence of aMTD907
VAIALAPVVVAP

SEQ ID NO: 238 - protein, 12 aa, Artificial Sequence
  Amino acid Sequence of aMTD908
VALALAPVVVAP

SEQ ID NO: 239 - protein, 12 aa, Artificial Sequence
  Amino acid Sequence of aMTD910
VAALLPAVVVAP

SEQ ID NO: 240 - protein, 12 aa, Artificial Sequence
  Amino acid Sequence of aMTD911
VALALPAVVVAP

SEQ ID NO: 241 - protein, 12 aa, Artificial Sequence
  Amino acid Sequence of aMTD912
VALLAPAVVVAP

SEQ ID NO: 242 - protein, 99 aa, Artificial Sequence
  Amino acid sequence of SD
MAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

SEQ ID NO: 243 - protein, 499 aa, Artificial Sequence
  Amino acid sequence of iCP-mParkin
AVALIVVPALAPQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDVMAEQSDKDVKYYTLEEIQKHKDSKSTWLILHHKVYDLTKFLEEHPGGEEVLGEQAGGDATENFEDVGHSTDARELSKTYIIGELHPDDRSKIAKPSETL

(19) United States
(12) Patent Application Publication
(10) Pub. No.: US 2023/0073000 A1
(43) Pub. Date: Mar. 9, 2023

(54) Improved Cell-Permeable Modified Parkin Recombinant Protein for Treatment of Neurodegenerative Diseases and Use Thereof

(71) Applicant: Cellivery Therapeutics, Inc., Seoul (KR)

(72) Inventor: Daewoong Jo, Brentwood, TN (US)

(21) Appl. No.: 17/798,139

(22) PCT Filed: Sep. 3, 2021
(86) PCT No.: PCT/KR2021/011974

§ 371 Date: Aug. 8, 2022

Related U.S. Application Data
(60) Provisional application No. 63/074,697, filed on Sep. 4, 2020.

(51) Int. Cl. C12N 9/00; C07K 1/18; C12N 15/63; A61K 38/53; A61P 25/00

(52) U.S. Cl. CPC C12N 9/93; C12Y 600/00; C07K 1/18; C12N 15/63; A61K 38/53; A61P 25/00

(57) ABSTRACT

Disclosed herein is iCP-mParkin. The iCP-mParkin exhibits biological features suitable for treating neuronal cell damage-related diseases. Thus, the iCP-mParkin provided herein can be used in a composition or method for treating, preventing, or alleviating Parkinson's disease, Alzheimer's disease, and Huntington's disease. Furthermore, the iCP-mParkin is higher in stability than conventional iCP-Parkin and as such, is suitable for use as a protein medicine. In addition, the iCP-mParkin obtained by the preparation method provided herein is of high purity and the preparation method is suitable for mass production.

### TECHNICAL FIELD

### Cross-Reference to Related Applications

This application claims the benefit of and priority to U. S. Provisional Patent Application Ser. No. 63/074,697, filled Sep. 4, 2020, the content of which is incorporated herein by reference in its entirety.

### TECHNICAL FIELD

The present invention relates to a pharmaceutical candidate that can fundamentally treat degenerative brain diseases (e.g., Parkinson's and Alzheimer's disease) by regenerating damaged neurons and by removing malignant protein aggregates along with damaged mitochondria accumulated in the brain. This invention is advanced in technology that modified the protein structure and purification method of the existing API manufacturing method and development in order to improve the API development method as a pharmaceutical substance for the purpose of clinical application.

### BACKGROUND ART

Neurodegenerative Diseases and their Unmet Medical Needs

Neurodegenerative disease (NDD) is a disease that occurs as the structure and function of the body gradually degenerate, especially in the brain and spinal cord. It causes an abnormal disability in learning, memory, etc. through the imbalance of the neurotransmitter. It can be classified by considering the main symptoms that appear and the brain regions that are affected, which includes Alzheimer's disease (AD), Parkinson's disease (PD), and Huntington's disease (HD). There is no suitable treatment so far that can be applied to neurogenesis nor to inhibit the apoptosis of neuronal cell.

Parkinson's Disease

PD is a degenerative disease caused by the selective loss of dopaminergic neuronal cells in the nigrostriatal system (dopamine system) between the substantia nigra pars compacta and the striatum. The global number of patients is about 10 million, and the number is increasing. PD is caused by reduced stimulation of the motor neuron cortex due to incomplete production of dopamine in the substantia nigra (SN). It is classified as familial PD caused by factors related to PD accompanied by clinical symptoms of movement disorders such as tremor, bradykinesia, and rigidity, and non-motor disorders such as depression, insomnia, and cognitive impairment. This suggests that the etiology of PD is a multiple system disease that causes the death of nerve cells in a wide range of nervous systems.

In the case of familial PD caused by genetic defects, α-Synuclein (PARK1/4), LRRK2 (PARKS), PINK1 (PARK6), Parkin (PARK2), DJ-1 (PARK7), etc. are well known. In addition, the genetic factors of familial PD are thought to play a very important role in sporadic PD, and PD causative genes such as Parkin which have its location at an important stage of pathogenesis. Loss of E3 ubiquitin ligase function due to Parkin mutation inhibits dopamine release results in accumulation of specific substrates or induces degeneration of dopaminergic neurons, leading to neuronal cell death. In addition, it is reported that Parkin has a protective function in mitochondria and exhibits a cytoprotective effect that reduces mitochondrial swelling and apoptosis caused by stress. Abnormal pathological protein aggregate accumulation induces the death of brain neurons by the corresponding mechanism and causes PD: mitochondria dysfunction and ubiquitin-proteasome system (UPS) dysfunction.

According to a recent pathological analysis of PD, α-Synuclein is initially induced in the olfactory bulb and enteric plexus, and is transported and translocated to the medulla oblongata and pons, which is then transmitted to PD. Afterwards, α-Synuclein is translocated to the midbrain, and then to the cerebral region including the limbic cortex, and various degenerative pathologies related to dementia also appear. When symptoms become severe, symptomatic drugs alone cannot stop the progression of PD.

The current treatment for PD only temporarily suppresses the symptoms, but it does not satisfy the safety and efficacy of the patients and by far it is impossible to improve the underlying condition that causes serious side effects. The size of the unmet demand is estimated at about $1.08 billion, and it is necessary to develop a new mechanism-specific disease modifying drug that can fundamentally treat the disease. Currently, biopharmaceuticals, such as antibody therapeutics, find it difficult to penetrate the blood-brain barrier (BBB) or transfer to the inside of brain neurons where pathological substances are generated. As the diagnosis technology for PD has been improving, the possibility of treating PD has increased. This will gradually increase the related treatment market. The development of therapeutics based on new pharmacological delivery technology is competitive and requires the development of mechanism-specific disease modifying therapeutics that can provide fundamental treatment.

Alzheimer's Disease

AD is the most common type of dementia, occurring in middle age to old age (over 65 years of age), accounting for about 60-70% of all dementias. Efforts have been made to find the causes of AD for a long time, but there still many unknowns. A key symptom found in AD patients is a decrease in memory and cognitive ability. In the early stages, the recent memory is impaired along with memory function deteriorated, and communication disorders appear. During the disease, psycho-behavioral symptoms such as anxiety, restlessness, depression, and delusions are often accompanied, and cognitive and neurological symptoms progress very slowly.

Neurofibrillary tangles, which are abnormally twisted in nerve cells, are also characteristic of AD. Phosphorylation of tau protein promotes nerve cell destruction through biochemical reactions. However, neurofibrillary tangles are not only found in the brains of patients with Alzheimer's dementia, but are also found in normal people, however the amount is large in the brains of patients with Alzheimer's dementia. It has been reported that the accumulation of damaged mitochondria and overactivation of the UPS in cells caused by neurotoxic substances such as oligomer Aβ or Neurofibrillary Tangles induce brain neuronal cell death. When UPS dysfunction occurs, abnormal Aβ and p-Tau proteins are aggregated in the cytoplasm of neurons, and AD is induced by neuronal cell death. However, the detailed molecular mechanism has not yet been elucidated.

The global dementia-related market is estimated to be USD 604 billion (about 1% of global GDP) as of 2010, and the number of patients is increasing not only in developed countries but also in developing countries, so it is urgent matter to obtain dementia-related therapeutics. In particular, the baby boomers of the United States will turn 65 on Jan. 1, 2011, and by Dec. 31, 202, 10,000 people will turn 65 every day and enter the elderly population. Dementia-related medical and nursing expenses in the United States are estimated to be approximately $226 billion annually, and if the current trend continues, dementia-related medical expenses are expected to reach $1.1 trillion by 2050.

AD, which accounts for more than 60% of the causes of dementia, progressively deteriorate cognitive functions such as memory, language ability, executive function, and movement ability. The pathological markers of Amyloid-β (Aβ) and Tau protein are not degraded in vivo when accumulated. The main cause of AD dementia is a degenerative brain disease in which cortical/hippocampal neurons die. Various risk factors such as other pathologies, genetic abnormalities, age, and causes that have not been identified yet still exist. As a pathological feature of AD, representative amyloid precursor protein (APP) produces Aβ through abnormal metabolism, and due to its poor solubility in water it aggregates and accumulates in the brain and forms Senile Plaques. The formed Senile Plaque has cytotoxicity and promotes the destruction of brain neuronal cells, and the neurotransmitter gets affected, causing clinical dementia.

Tau protein also undergoes a biochemical reaction to form neurofibrillary tangles in an abnormally twisted form during phosphorylation, and at the same time has neurotoxicity (Cell Toxicity), thus promoting the destruction of nerve cells in the brain. However, although neurofibrillary tangles are found in normal state, the amount is particularly high in the brains of AD patients. It has been reported that the accumulation of damaged mitochondria and overactivation of the UPS in cells caused by neurotoxic substances such as oligomer Aβ or Neurofibrillary Tangles induce brain neuronal cell death. When UPS dysfunction occurs, abnormal Aβ and p-Tau proteins are aggregated in the cytoplasm of neurons, and AD is induced by neuronal cell death. However, the detailed molecular mechanism has not yet been elucidated. Therefore, it is necessary to develop a novel mechanism for treatment of dementia in accordance with the market demand.

Development of Improved Cell-Permeable Parkin (iCP-Parkin)

Parkin is a protein which anti-apoptotic effect on neuronal cell death has been demonstrated and proven in basic research. In animal models of PD, there is a report that when Parkin is supplemented (replenished), neuronal cells go through reactivation and the symptoms of PD are treated. Demonstrating evidences that Parkin can act as a fundamental therapeutic agent for PD by activating inactivated dopaminergic neuronal cells. It is known that Parkin-induced mitophagy plays an important mechanism as it has been found that Parkin's function loss is a pathological factor in PD (J. Cell Biol. 2008). Parkin is an E3 ubiquitin ligase that removes damaged mitochondria and/or induces mitophagy (Nat Commun, 2012).

When the Parkin or PINK1 gene is removed from the C. elegans PD model, mitochondrial dysfunction and motor function are lost, and a defective phenotype caused by PINK1 mutation in Parkin-expressed PINK1 deficiency Drosophila is reported to be restored by replenishment of Parkin (Nature, 2006). In addition, it was reported that there is a problem in Parkin expression in the brain of patients with PD (Nat Med, 2005). In addition, the main pathogenesis of PD is accumulation of α-Synuclein present in the brain due to malfunction, which, combined with additional mitochondrial complex I degradation, oxidative stress, and ubiquitin proteasome inhibition, leads to gradual apoptosis of dopaminergic neuronal cells. Parkin mutation induces accumulation of abnormal protein caused by loss of E3 ubiquitin ligase activity, inhibition of dopamine release and degeneration of dopaminergic neuronal cells, causing neuronal cell deaths. In other words, abnormal Parkin function can cause accumulation of α-Synuclein.

In order to derive an optimized candidate for PD targeting candidate using the TSDT platform, iCP-Parkin was developed through the following structural screening process.

Randomly selected aMTD321 sequences and fused to solubilization domains [SDA (184 A/a) derived from Protein S of Myxococcus xanthus or SDB (99 A/a) derived from cytochrome b of Rattus norvegicus] His-aMTD321-Parkin-SDA (HM321PSA) and His-aMTD321-Parkin-SDB (HM321PSB) were derived. (2) The fusion of aMTD321 and SDB combination was determined as the basic backbone structure. (3) Optimal aMTD Screening Process: Through substitution of aMTD sequences, we compare and analyze the solubility, yield, cell-permeability, and biological activity of recombinant protein to determine the optimal aMTD (aMTD524). 10 types of aMTDs were screened with a basic structure combining Parkin and solubilization domain B (SDB). Among 10 types of aMTD, aMTD524 had the best solubility and yield.

In addition, as a result of verifying the cell-permeability of 10 aMTD-fusion Parkin recombinant proteins, all proteins had cell permeability and aMTD524 was the 4th best. In addition, it was verified that aMTD524 had the best cytoprotective effect in the cytotoxic environment induced by 6-OHDA, a neurotoxin. (4) Comparison with other CPPs (TAT, PolyR, DPV03) which proves the superiority of aMTD-fused Parkin. (5) Removed His-Tag and demonstrated the equivalence. (6) aMTD524-Parkin-SDB (M524PSB) was determined as the lead of iCP-Parkin.

In the cytotoxic environment induced by another neurotoxin, MPP⁺, aMTD524 had the third cytoprotective effect, and in the annexin V experiment, which can stain apoptotic cells in the cytotoxic environment induced by 6-OHDA, aMTD524 has the best cytoprotective effect. Based on this, the aMTD524/SDB-fusion human Parkin recombinant protein was selected by optimized lead structure. This structure was first determined as improved Cell-Permeable Parkin (iCP-Parkin), a mechanism-specific PD-targeted therapeutics. That is, iCP-Parkin is a recombinant protein with high cell permeability made by binding aMTD524 (AVALIVVPALAP (SEQ ID No.123)) to the functional domain of Parkin protein, which has cytoprotective effect.

### Reference

1. PCT/KR2016/008174

### DISCLOSURE OF INVENTION

### Technical Problem

As described in the prior section, iCP-Parkin, the prior art, has great potential showing neuroprotective activity and anti-PD efficacy. However, in order to produce iCP-Parkin as a medicinal product for the development of a novel therapeutic biologics, it is necessary to increase the production yield and develop an effective process that can be mass-produced. For this, the present invention, as an advanced system of the prior art, includes 1) to modify the structure of the iCP-Parkin (i.e., iCP-mParkin) in order to improve protein stability, and also 2) to develop a proper purification process of proteins, capable of manufacturing in a large-scale, with maintaining the therapeutic activities and action modes of the prior art.

### Solution to Problem

The present disclosure provides an iCP (improved cell-permeable)—mParkin recombinant protein.

According to one embodiment, the recombinant protein comprises:

i) a modified Parkin protein; and

ii) an advanced macromolecule transduction domain (aMTD),

wherein, the modified Parkin protein has an amino acid sequence of SEQ ID No: 1,

the aMTD has an amino acid sequence selected from the group consisting of SEQ ID No: 2-241.

According to one embodiment, the recombinant protein further comprises one or more solubilization domain (SD)(s).

According to one embodiment, the recombinant protein is represented by any one of the following structural formulae:

A-B, B-A, A-B-C, A-C-B, B-A-C, B-C-A, C-A-B, C-B-A and A-C-B-C

wherein A is an advanced macromolecule transduction domain (aMTD),

B is a modified Parkin protein,

and C is a solubilization domain (SD).

According to one embodiment, the recombinant protein has an amino acid sequence of SEQ ID NO:243.

According to one embodiment, the SD(s) have an amino acid sequence of SEQ ID NO:242.

According to one embodiment, the recombinant protein is used for treating neurodegenerative disease

wherein the neurodegenerative disease comprises Parkinson's disease, Alzheimer's disease, and Huntington's disease.

According to one embodiment, a polynucleotide sequence encoding the iCP-mParkin recombinant protein is provided.

According to one embodiment, a recombinant expression vector comprising the polynucleotide sequence is provided.

According to one embodiment, a transformant transformed with the recombinant expression vector is provided.

According to one embodiment, a composition comprising the iCP-mParkin recombinant protein as an active ingredient is provided.

Provided according to one embodiment is a pharmaceutical composition for treating neurodegenerative disease, comprising the iCP-mParkin recombinant protein as an active ingredient; and a pharmaceutically acceptable carrier is provided.

In this regard, the neurodegenerative disease comprises Parkinson's disease, Alzheimer's disease, and Huntington's disease.

Provided according to one embodiment is a use of the iCP-mParkin recombinant protein as a medicament for treating neurodegenerative disease.

In the regard, the neurodegenerative disease comprises Parkinson's disease, Alzheimer's disease, and Huntington's disease.

According to one embodiment, a medicament comprising the iCP-mParkin recombinant protein is provided.

Provided according to one embodiment is a use of the iCP-mParkin recombinant protein for the preparation of a medicament for treating neurodegenerative disease.

In this regard, the neurodegenerative disease comprises Parkinson's disease, Alzheimer's disease, and Huntington's disease.

Provided according to one embodiment is a method of treating neurodegenerative disease in a subject.

In this regard, the method comprises administering to the subject a therapeutically effective amount of the iCP-mParkin recombinant protein.

According to one embodiment, a method for preparing the iCP-mParkin recombinant protein is provided.

In this regard, the method comprises:

preparing the recombinant expression vector comprising a polynucleotide sequence encoding the iCP-mParkin recombinant protein;

preparing a transformant using the recombinant expression vector;

culturing the transformant; and

obtaining the recombinant protein expressed by the culturing.

According to one embodiment, the obtaining the recombinant protein comprises:

washing of an inclusion body;

performing the first ion exchange chromatography; and

performing the second ion exchange chromatography.

According to one embodiment, the washing comprises one step washing using pH 8 washing buffer.

According to one embodiment, the first ion exchange chromatography is a cation exchange chromatography, and

the second ion exchange chromatography is an anion exchange chromatography.

According to one embodiment, the second ion exchange chromatography comprises:

washing in an 8.0 mS/Cm conductivity condition, and

elution in a 9.0 mS/Cm conductivity condition.

According to one embodiment, the culturing comprises a fed-batch fermentation.

### Advantageous Effects of Invention

With process development and structure modification, iCP-mParkin can solve several previous limitation and issues (e.g., low stability & monomer yield with heterogeneity, and high-cost SEC usage) of the prior art iCP-Parkin, thus leading to be developed as an advanced drug material with a benefit of a powerful therapeutic potential of iCP-Parkin, which is already proven in peer-review journal publication. iCP-mParkin, a modified structure of iCP-Parkin with the Ubl domain deleted, was selected as the final structure of iCP-Parkin based on its superior purity, homogeneity, stability, and biological activity.

### BRIEF DESCRIPTION OF DRAWINGS

FIG. 1 shows the structure of Parkin protein and hydrophobicity.

FIG. 2 shows structure screening of iCP-Parkin variants.

FIG. 3 shows results from protein analysis of iCP-Parkin variants using HPLC analysis.

FIG. 4 shows a list of structures of iCP-Parkin variants with cysteine-to-serine alteration.

FIG. 5 shows structure diagrams of iCP-Parkin (prior art) and iCP-mParkin.

FIG. 6 illustrates development of Improved Process (IP) to purify recombinant proteins with higher homogeneity and purity.

FIG. 7 is a diagram describing that impurity of produced proteins was decreased by IB washing.

FIG. 8 shows the modified elution method to remove impurity of recombinant proteins.

FIG. 9 shows development of one-step IB washing and 2-Step column purification with an optimized elution method to obtain a higher portion of monomeric iCP-mParkin.

FIG. 10 is view describing that 2-step column purification process effectively removes impurities.

FIG. 11 shows incorporation of step-elution for large-scale production of monomeric iCP-mParkin.

FIG. 12 shows the final process (FP) of protein manufacturing for preclinical and clinical development of iCP-mParkin.

FIG. 13 shows chromatogram comparison of iCP-Parkin and iCP-mParkin purified under identical conditions.

FIG. 14 shows improved stability of iCP-mParkin.

FIG. 15 shows stability of iCP-mParkin at 37° C. over the span of 48 hours and at 25° C. over the span of 4 days.

FIG. 16 shows stability of iCP-mParkin depending on concentration.

FIG. 17 shows characterization summary of iCP-mParkin: iCP-mParkin with structural stability applicable at clinical development level.

FIG. 18 shows results of fed-batch fermentation.

FIG. 19 shows comparison on IB yield of iCP-mParkin depending on cell mass cultivation.

FIG. 20 shows purification results using iCP-mParkin cell mass produced from fed-batch fermentation.

FIG. 21 shows cell line development for iCP-mParkin expression.

FIG. 22 shows a final result set of the cell line development at CMO.

FIG. 23 shows iCP-mParkin produced at CMO by final process (FP) method.

FIG. 24 shows purity of iCP-mParkin produced at CMO and in-house (Cellivery).

FIG. 25 shows freezing/thawing and thermal stability of iCP-mParkin produced at CMO.

FIG. 26 shows demonstration on comparable biological activity of iCP-mParkin produced at CMO and in-house (Cellivery).

FIG. 27 shows that iCP-mParkin is cell-permeable.

FIG. 27 shows visualized cell-permeability of iCP-Parkin and iCP-mParkin Recombinant Proteins. C2C12 cells were treated with FITC-labeled proteins (10 μM) fused to aMTD for 2 hour at 37° C. Cell-permeability of the proteins was visualized by laser scanning confocal microscopy (A). Determination of Cell-Permeability of iCP-Parkin and iCP-mParkin Recombinant Proteins by flow cytometry. The cell-permeability of both Parkin recombinant proteins are visually compared each other in C2C12 (B) and A549 (C). White bar represents untreated cells (vehicle); black bar line represents the cells treated with equal molar concentration of FITC (FITC only); blue bar indicates the cells treated with FITC-labeled iCP-Parkin; and red bar indicates the cells treated with FITC-labeled iCP-mParkin. The cell-permeability was determined by flow cytometry analysis.

FIG. 28 shows that iCP-mParkin is cell-permeable in damaged cells.

FIG. 28 shows determination of Cell-Permeability of iCP-Parkin and iCP-mParkin Recombinant Proteins. The cell-permeability of both Parkin recombinant proteins was visually compared each other in C2C12 after treatment of 6-OHDA. After 2 hours incubation, cells were lysed and analyzed by western blot analysis.

FIG. 29 shows results from auto-ubiquitination assay of Parkin recombinant proteins.

FIG. 29 shows results from in vitro auto-ubiquitination activity of iCP-Parkin and iCP-mParkin with/without ATP. Auto-ubiquitination was assessed by western blot analyze with anti-ubiquitin (FK2).

FIG. 30 shows results from analysis of cell viability by iCP-Parkin and iCP-mParkin.

SH-SY5Y cell cells were treated with 30 μM 6-OHDA and 10 μM iCP-Parkin or iCP-mParkin. After 24 hours incubation, cells were subjected to ATP Glo assay (A). Note that cell viability by two recombinant proteins is almost the same. (B) Changes of cellular morphology from the treatments were monitored by light microscopy

FIG. 31 shows that iCP-mParkin promotes mitophagy under mitochondria damaged condition.

(A) SH-SY5Y cells were incubated with CCCP and iCP-Parkin/iCP-mParkin for 4 hours. Western blot analysis for detecting LC3B-II, an autophagy marker, in lysates from CCCP or CCCP+iCP-mParkin treated SH-SY5Y cell under the treatment of chloroquine, an autophagy inhibitor. (B) Confocal microscope images for detecting mitophagy.

FIG. 32 shows that iCP-mParkin promotes mitochondria biogenesis and suppress ROS generation under mitochondria damaged condition.

FIG. 33 shows demonstration on comparable MoA1 of iCP-mParkin and iCP-Parkin.

FIG. 34 shows that iCP-mParkin suppresses sodium arsenide-induced cell death and aggregated forms of α-Synuclein.

Sodium arsenide is toxic to TagGFP2-α-Synuclein SH-SY5Y cells and induces the accumulation of aggregated α-Synuclein (A, B). ELISA analysis showing significant decrease of pathological α-Synuclein forms such as oligomeric and filamentous α-Synuclein by iCP-mParkin in soluble fraction at 8 hours.

FIG. 35 shows demonstration on comparable MoA2 of iCP-mParkin and iCP-Parkin.

FIG. 36 shows that iCP-mParkin did not show in vivo toxicity compared to iCP-Parkin.

iCP-Parkin and iCP-mParkin (60 mg/kg) was intravenously injected 3 times per week for 2 weeks. Body weight, fur condition, and behavior of mice were analyzed.

FIG. 37 shows that iCP-mParkin did not show in vivo toxicity compared to iCP-Parkin.

iCP-Parkin and iCP-mParkin (60 mg/kg) was intravenously injected 3 times per week for 2 weeks. The ratio of spleen weight vs. body weight from treated mice were analyzed.

FIG. 38 shows that iCP-mParkin ameliorates behavioral and molecular defects in 6-OHDA-induced PD animal models similar to iCP-Parkin.

FIG. 38 shows an efficacy of iCP-Parkin in a 6-OHDA-induced Parkinson's disease (PD) mouse model. 6-OHDA (4 μg/head) was injected into the right side of the striatum. Rota-rod test. Relative behavior activity is based on the value of the diluent control as 100%.

FIG. 39 shows that iCP-mParkin ameliorates behavioral and molecular defects in 6-OHDA-induced PD animal models.

A shows a schematic diagram of the experimental protocol. 6-OHDA (4 μg/head) was injected into the right side of the the striatum. iCP-mParkin was i.v. injected 3 times per week for 4 weeks from 2 weeks after injecting 6-OHDA into the ST on the right side of the brain. B shows a Rota-rod test. Relative behavior activity is based on the value of the diluent control as 100%.

FIG. 40 shows iCP-mParkin ameliorates behavioral and molecular defects in 6-OHDA-induced PD animal models.

FIG. 40 shows a western blot analysis of tyrosine hydroxylase (TH) expression and graph of relative TH expression quantified using ImageJ. L and R indicate the left and right sides of the brain, respectively.

FIG. 41 shows improving cognitive function of iCP-mParkin in AD mouse model after 2 weeks of administration.

A shows an experiment design of iCP-mParkin dose-dependent in AD model for 2 weeks. B shows an administration of iCP-mParkin significantly improved cognitive function in a dose-dependent manner in AD model

FIG. 42 shows improving cognitive function of iCP-mParkin in AD mouse model after 4 weeks of administration.

A shows an experiment design of iCP-mParkin dose-dependent in AD model for 4 weeks. B shows an administration of iCP-mParkin significantly improved cognitive function in a low dose-dependent manner in AD model.

FIG. 43 shows that in the brain of AD model, iCP-mParkin eliminates pathological proteins.

(A) Representative immunohistochemistry images show a removes amyloid-beta (Aβ) plaque by iCP-mParkin and neuroprotective effect. (B) Representative dot blot images showing a significant decrease in pathological Aβ plaque forms by iCP-mParkin in the soluble fraction. (C) Quantification of dot blot images showing a significant decrease in Aβ plaques ratio by iCP-mParkin.

FIG. 44 shows that iCP-mParkin blocked relative oxidative stress (ROS) accumulation at 3 & 6 hour in Aβ treated HT22 cell.

FIG. 45 shows that iCP-mParkin had high brain delivery in the AD model by LCMS/MS analysis.

(A) In the brain of AD model, Quantified amounts of iCP-mParkin is more abundant than Non-CP-mParkin. (B) The maximal brain delivery of iCP-mParkin in the AD model was 2.9%. Time point: 0.5 and 1 hour. **p<0.01, data is the means ±S.E.M with Student's t-test, respectively.

### MODE FOR THE INVENTION

Unless otherwise defined, all technical and scientific terms used herein have the same meaning as commonly understood by experts in the art to which the present invention belongs. All the publications, patents, and other documents cited in the description are incorporated by reference in their entireties.

Additionally, unless specifically stated throughout the specification, the terms “comprising”, “including”, or “containing” are intended to designate including any component (or constituent element) without particular limitations thereto, and cannot be construed as excluding the addition of a different component (or constituent element).

As used herein, the term “amino acid” is intended to encompass D-amino acids and chemically modified amino acids in a broad sense as well as naturally occurring L aamino acids or residues thereof. For example, the amino acid mimetics and analogs fall within the scope of the amino acid. Herein, the mimetics and analogs may include functional equivalents thereof.

As used herein, the term “prevention” means all actions that are performed to suppress or delay the onset of neurodegenerative disease by administering the iCP-mParkin recombinant protein according to the present disclosure, and the term “treatment” means all actions that are performed to alleviate or beneficially change symptoms of neurodegenerative disease by administering the iCP-mParkin recombinant protein.

As used herein, the term “administration” refers to the delivery of a pharmaceutical composition according to the present disclosure into a subject in any suitable manner.

As used herein, the term “subject” refers to any animal including humans, which has suffered from or is at risk for neurodegenerative disease. Examples of the animal, which is in need of treating neurodegenerative disease or symptoms thereof, include cattle, horses, sheep, swine, goats, camels, antelope, dogs, and cats, but are not limited thereto.

I. iCP (Improved Cell-Permeable)—mParkin Recombinant Protein

1. Modified Parkin

One embodiment of the present disclosure provides an iCP (improved cell-permeable)—mParkin recombinant protein comprising modified Parkin. The modified Parkin brings about improved stability in the protein, compared to conventional iCP-Parkin.

The modified Parkin may be in a truncated form resulting from removal of a domain from Parkin protein. In one embodiment, the truncated form may result from removal of at least one domain selected from the group consisting of Ubl, RING0, RING1, IBR, and RING2 of Parkin protein. In an exemplary embodiment, the modified parkin may be in a form lacking Ubl domain. In a more exemplary embodiment, the modified parkin may have the amino acid sequence of QEMNATGGDDPRNAAGGCEREPQSLTRVDLSSSVLPGDSVGLAVILHTDSRKDSPPAGSPAGRSIYNSFYV YCKGPCQRVQPGKLRVQCSTCRQATLTLTQGPSCWDDVLIPNRMSGECQSPH CPGTSAEFFFKCGAHPTSDKETSVALHLIATNSRNITCITCTDVRSPVLVFQCNS RHVICLDCFHLYCVTRLNDRQFVHDPQLGYSLPCVAGCPNSLIKELHHFRILGE EQYNRYQQYGAEECVLQMGGVLCPRPGCGAGLLPEPDQRKVTCEGGNGLGC GFAFCRECKEAYHEGECSAVFEASGTTTQAYRVDERAAEQARWEAASKETIK KTTKPCPRCHVPVEKNGGCMHMKCPQPQCRLEWCWNCGCEWNRVCMGDH WFDV (SEQ ID No: 1). However, the modified parkin is not limited to the amino acid sequence of SEQ ID NO: 1, but include any variant that exhibits an identical or similar effect to the sequence.

2. Domain that Facilitates Delivery of a Bioactive Molecule into Cells Across their Plasma Membranes

The present disclosure provides a iCP (improved cell-permeable)—mParkin recombinant protein comprising a domain that facilitates a bioactive molecule into cells across their plasma membranes. The domain that facilitates the delivery of a bioactive molecule into cells across their plasma membranes may be exemplified by an aMTD domain, but with no limitations thereto, and may include cationic, chimeric, hydrophobic CPP (cell penetrating peptide).

As for the bioactive molecule, their examples include proteins, peptides, nucleic acids, compounds, and so on. In the present disclosure, the aMTD domain may mean a peptide that facilitates the delivery of the above-described mParkin protein across plasma membranes. With respect to the aMTD domain, reference may be made to Korean Patent Number 10-1971021, the content of which is incorporated herein by reference in its entirety.

In one embodiment, the aMTD domain may include the amino acid sequence selected from the group SEQ ID NOS: 2 to 241. In an exemplary embodiment, the aMTD domain may include the amino acid sequence of SEQ ID NO: 123.

TABLE 1
aMTD Domain

| SEQ | |
|---|---|
| ID | |
| No. | Sequence |
| 2 | Ala Ala Ala Leu Ala Pro Val Val Leu Ala Leu Pro |
| 3 | Ala Ala Ala Val Pro Leu Leu Ala Val Val Val Pro |
| 4 | Ala Ala Leu Leu Val Pro Ala Ala Val Leu Ala Pro |
| 5 | Ala Leu Ala Leu Leu Pro Val Ala Ala Leu Ala Pro |
| 6 | Ala Ala Ala Leu Leu Pro Val Ala Leu Val Ala Pro |
| 7 | Val Val Ala Leu Ala Pro Ala Leu Ala Ala Leu Pro |
| 8 | Leu Leu Ala Ala Val Pro Ala Val Leu Leu Ala Pro |
| 9 | Ala Ala Ala Leu Val Pro Val Val Ala Leu Leu Pro |
| 10 | Ala Val Ala Leu Leu Pro Ala Leu Leu Ala Val Pro |
| 11 | Ala Val Val Leu Val Pro Val Leu Ala Ala Ala Pro |
| 12 | Val Val Leu Val Leu Pro Ala Ala Ala Ala Val Pro |
| 13 | Ile Ala Leu Ala Ala Pro Ala Leu lie Val Ala Pro |
| 14 | Ile Val Ala Val Ala Pro Ala Leu Val Ala Leu Pro |
| 15 | Val Ala Ala Leu Pro Val Val Ala Val Val Ala Pro |
| 16 | Leu Leu Ala Ala Pro Leu Val Val Ala Ala Val Pro |
| 17 | Ala Leu Ala Val Pro Val Ala Leu Leu Val Ala Pro |
| 18 | Val Ala Ala Leu Pro Val Leu Leu Ala Ala Leu Pro |
| 19 | Val Ala Leu Leu Ala Pro Val Ala Leu Ala Val Pro |
| 20 | Ala Ala Leu Leu Val Pro Ala Leu Val Ala Val Pro |
| 21 | Ala Ile Val Ala Leu Pro Val Ala Val Leu Ala Pro |
| 22 | Ile Ala Ile Val Ala Pro Val Val Ala Leu Ala Pro |
| 23 | Ala Ala Leu Leu Pro Ala Leu Ala Ala Leu Leu Pro |
| 24 | Ala Val Val Leu Ala Pro Val Ala Ala Val Leu Pro |
| 25 | Leu Ala Val Ala Ala Pro Leu Ala Leu Ala Leu Pro |
| 26 | Ala Ala Val Ala Ala Pro Leu Leu Leu Ala Leu Pro |
| 27 | Leu Leu Val Leu Pro Ala Ala Ala Leu Ala Ala Pro |
| 28 | Leu Val Ala Leu Ala Pro Val Ala Ala Val Leu Pro |
| 29 | Leu Ala Leu Ala Pro Ala Ala Leu Ala Leu Leu Pro |
| 30 | Ala Leu Ile Ala Ala Pro Ile Leu Ala Leu Ala Pro |
| 31 | Ala Val Val Ala Ala Pro Leu Val Leu Ala Leu Pro |
| 32 | Leu Leu Ala Leu Ala Pro Ala Ala Leu Leu Ala Pro |
| 33 | Ala Ile Val Ala Leu Pro Ala Leu Ala Leu Ala Pro |
| 34 | Ala Ala Ile Ile Val Pro Ala Ala Leu Leu Ala Pro |
| 35 | Ile Ala Val Ala Leu Pro Ala Leu Ile Ala Ala Pro |
| 36 | Ala Val Ile Val Leu Pro Ala Leu Ala Val Ala Pro |
| 37 | Ala Val Leu Ala Val Pro Ala Val Leu Val Ala Pro |
| 38 | Val Leu Ala Ile Val Pro Ala Val Ala Leu Ala Pro |
| 39 | Leu Leu Ala Val Val Pro Ala Val Ala Leu Ala Pro |
| 40 | Ala Val Ile Ala Leu Pro Ala Leu Ile Ala Ala Pro |
| 41 | Ala Val Val Ala Leu Pro Ala Ala Leu Ile Val Pro |
| 42 | Leu Ala Leu Val Leu Pro Ala Ala Leu Ala Ala Pro |
| 43 | Leu Ala Ala Val Leu Pro Ala Leu Leu Ala Ala Pro |
| 44 | Ala Leu Ala Val Pro Val Ala Leu Ala Ile Val Pro |
| 45 | Ala Leu Ile Ala Pro Val Val Ala Leu Val Ala Pro |
| 46 | Leu Leu Ala Ala Pro Val Val Ile Ala Leu Ala Pro |
| 47 | Leu Ala Ala Ile Val Pro Ala Ile Ile Ala Val Pro |
| 48 | Ala Ala Leu Val Leu Pro Leu Ile Ile Ala Ala Pro |
| 49 | Leu Ala Leu Ala Val Pro Ala Leu Ala Ala Leu Pro |
| 50 | Leu Ile Ala Ala Leu Pro Ala Val Ala Ala Leu Pro |
| 51 | Ala Leu Ala Leu Val Pro Ala Ile Ala Ala Leu Pro |
| 52 | Ala Ala Ile Leu Ala Pro Ile Val Ala Leu Ala Pro |
| 53 | Ala Leu Leu Ile Ala Pro Ala Ala Val Ile Ala Pro |
| 54 | Ala Ile Leu Ala Val Pro Ile Ala Val Val Ala Pro |
| 55 | Ile Leu Ala Ala Val Pro Ile Ala Leu Ala Ala Pro |
| 56 | Val Ala Ala Leu Leu Pro Ala Ala Ala Val Leu Pro |
| 57 | Ala Ala Ala Val Val Pro Val Leu Leu Val Ala Pro |
| 58 | Ala Ala Leu Leu Val Pro Ala Leu Val Ala Ala Pro |
| 59 | Ala Ala Val Leu Leu Pro Val Ala Leu Ala Ala Pro |
| 60 | Ala Ala Ala Leu Ala Pro Val Leu Ala Leu Val Pro |
| 61 | Leu Val Leu Val Pro Leu Leu Ala Ala Ala Ala Pro |
| 62 | Ala Leu Ile Ala Val Pro Ala Ile Ile Val Ala Pro |
| 63 | Ala Leu Ala Val Ile Pro Ala Ala Ala Ile Leu Pro |
| 64 | Leu Ala Ala Ala Pro Val Val Ile Val Ile Ala Pro |
| 65 | Val Leu Ala Ile Ala Pro Leu Leu Ala Ala Val Pro |
| 66 | Ala Leu Ile Val Leu Pro Ala Ala Val Ala Val Pro |
| 67 | Val Leu Ala Val Ala Pro Ala Leu Ile Val Ala Pro |
| 68 | Ala Ala Leu Leu Ala Pro Ala Leu Ile Val Ala Pro |
| 69 | Ala Leu Ile Ala Pro Ala Val Ala Leu Ile Val Pro |
| 70 | Ala Ile Val Leu Leu Pro Ala Ala Val Val Ala Pro |
| 71 | Val Ile Ala Ala Pro Val Leu Ala Val Leu Ala Pro |
| 72 | Leu Ala Leu Ala Pro Ala Leu Ala Leu Leu Ala Pro |
| 73 | Ala Ile Ile Leu Ala Pro Ile Ala Ala Ile Ala Pro |
| 74 | Ile Ala Leu Ala Ala Pro Ile Leu Leu Ala Ala Pro |
| 75 | Ile Val Ala Val Ala Leu Pro Ala Leu Ala Val Pro |
| 76 | Val Val Ala Ile Val Leu Pro Ala Leu Ala Ala Pro |
| 77 | Ile Val Ala Val Ala Leu Pro Val Ala Leu Ala Pro |
| 78 | Ile Val Ala Val Ala Leu Pro Ala Ala Leu Val Pro |
| 79 | Ile Val Ala Val Ala Leu Pro Ala Val Ala Leu Pro |
| 80 | Ile Val Ala Val Ala Leu Pro Ala Val Leu Ala Pro |
| 81 | Val Ile Val Ala Leu Ala Pro Ala Val Leu Ala Pro |
| 82 | Ile Val Ala Val Ala Leu Pro Ala Leu Val Ala Pro |
| 83 | Ala Leu Leu Ile Val Ala Pro Val Ala Val Ala Pro |
| 84 | Ala Val Val Ile Val Ala Pro Ala Val Ile Ala Pro |
| 85 | Ala Val Leu Ala Val Ala Pro Ala Leu Ile Val Pro |
| 86 | Leu Val Ala Ala Val Ala Pro Ala Leu Ile Val Pro |
| 87 | Ala Val Ile Val Val Ala Pro Ala Leu Leu Ala Pro |
| 88 | Val Val Ala Ile Val Leu Pro Ala Val Ala Ala Pro |
| 89 | Ala Ala Ala Leu Val Ile Pro Ala Ile Leu Ala Pro |
| 90 | Val Ile Val Ala Leu Ala Pro Ala Leu Leu Ala Pro |
| 91 | Val Ile Val Ala Ile Ala Pro Ala Leu Leu Ala Pro |
| 92 | Ile Val Ala Ile Ala Val Pro Ala Leu Val Ala Pro |
| 93 | Ala Ala Leu Ala Val Ile Pro Ala Ala Ile Leu Pro |
| 94 | Ala Leu Ala Ala Val Ile Pro Ala Ala Ile Leu Pro |
| 95 | Ala Ala Ala Leu Val Ile Pro Ala Ala Ile Leu Pro |
| 96 | Leu Ala Ala Ala Val Ile Pro Ala Ala Ile Leu Pro |
| 97 | Leu Ala Ala Ala Val Ile Pro Val Ala Ile Leu Pro |
| 98 | Ala Ala Ile Leu Ala Ala Pro Leu Ile Ala Val Pro |
| 99 | Val Val Ala Ile Leu Ala Pro Leu Leu Ala Ala Pro |
| 100 | Ala Val Val Val Ala Ala Pro Val Leu Ala Leu Pro |
| 101 | Ala Val Val Ala Ile Ala Pro Val Leu Ala Leu Pro |
| 102 | Ala Leu Ala Ala Leu Val Pro Ala Val Leu Val Pro |
| 103 | Ala Leu Ala Ala Leu Val Pro Val Ala Leu Val Pro |
| 104 | Leu Ala Ala Ala Leu Val Pro Val Ala Leu Val Pro |
| 105 | Ala Leu Ala Ala Leu Val Pro Ala Leu Val Val Pro |
| 106 | Ile Ala Ala Val Ile Val Pro Ala Val Ala Leu Pro |
| 107 | Ile Ala Ala Val Leu Val Pro Ala Val Ala Leu Pro |
| 108 | Ala Val Ala Ile Leu Val Pro Leu Leu Ala Ala Pro |
| 109 | Ala Val Val Ile Leu Val Pro Leu Ala Ala Ala Pro |
| 110 | Ile Ala Ala Val Ile Val Pro Val Ala Ala Leu Pro |
| 111 | Ala Ile Ala Ile Ala Ile Val Pro Val Ala Leu Pro |
| 112 | Ile Leu Ala Val Ala Ala Ile Pro Val Ala Val Pro |
| 113 | Ile Leu Ala Ala Ala Ile Ile Pro Ala Ala Leu Pro |
| 114 | Leu Ala Val Val Leu Ala Ala Pro Ala Ile Val Pro |
| 115 | Ala Ile Leu Ala Ala Ile Val Pro Leu Ala Val Pro |
| 116 | Val Ile Val Ala Leu Ala Val Pro Ala Leu Ala Pro |
| 117 | Ala Ile Val Ala Leu Ala Val Pro Val Leu Ala Pro |
| 118 | Ala Ala Ile Ile Ile Val Leu Pro Ala Ala Leu Pro |
| 119 | Leu Ile Val Ala Leu Ala Val Pro Ala Leu Ala Pro |
| 120 | Ala Ile Ile Ile Val Ile Ala Pro Ala Ala Ala Pro |
| 121 | Leu Ala Ala Leu Ile Val Val Pro Ala Val Ala Pro |
| 122 | Ala Leu Leu Val Ile Ala Val Pro Ala Val Ala Pro |
| 123 | Ala Val Ala Leu Ile Val Val Pro Ala Leu Ala Pro |
| 124 | Ala Leu Ala Ile Val Val Ala Pro Val Ala Val Pro |
| 125 | Leu Leu Ala Leu Ile Ile Ala Pro Ala Ala Ala Pro |
| 126 | Ala Leu Ala Leu Ile Ile Val Pro Ala Val Ala Pro |
| 127 | Leu Leu Ala Ala Leu Ile Ala Pro Ala Ala Leu Pro |
| 128 | Ile Val Ala Leu Ile Val Ala Pro Ala Ala Val Pro |
| 129 | Val Val Leu Val Leu Ala Ala Pro Ala Ala Val Pro |
| 130 | Ala Ala Val Ala Ile Val Leu Pro Ala Val Val Pro |
| 131 | Ala Leu Ile Ala Ala Ile Val Pro Ala Leu Val Pro |
| 132 | Ala Leu Ala Val Ile Val Val Pro Ala Leu Ala Pro |
| 133 | Val Ala Ile Ala Leu Ile Val Pro Ala Leu Ala Pro |
| 134 | Val Ala Ile Val Leu Val Ala Pro Ala Val Ala Pro |
| 135 | Val Ala Val Ala Leu Ile Val Pro Ala Leu Ala Pro |
| 136 | Ala Val Ile Leu Ala Leu Ala Pro Ile Val Ala Pro |
| 137 | Ala Leu Ile Val Ala Ile Ala Pro Ala Leu Val Pro |
| 138 | Ala Ala Ile Leu Ile Ala Val Pro Ile Ala Ala Pro |
| 139 | Val Ile Val Ala Leu Ala Ala Pro Val Leu Ala Pro |
| 140 | Val Leu Val Ala Leu Ala Ala Pro Val Ile Ala Pro |
| 141 | Val Ala Leu Ile Ala Val Ala Pro Ala Val Val Pro |
| 142 | Val Ile Ala Ala Val Leu Ala Pro Val Ala Val Pro |
| 143 | Ala Leu Ile Val Leu Ala Ala Pro Val Ala Val Pro |
| 144 | Val Ala Ala Ala Ile Ala Leu Pro Ala Ile Val Pro |
| 145 | Ile Leu Ala Ala Ala Ala Ala Pro Leu Ile Val Pro |
| 146 | Leu Ala Leu Val Leu Ala Ala Pro Ala Ile Val Pro |
| 147 | Ala Leu Ala Val Val Ala Leu Pro Ala Ile Val Pro |
| 148 | Ala Ala Ile Leu Ala Pro Ile Val Ala Ala Leu Pro |
| 149 | Ile Leu Ile Ala Ile Ala Ile Pro Ala Ala Ala Pro |
| 150 | Leu Ala Ile Val Leu Ala Ala Pro Val Ala Val Pro |
| 151 | Ala Ala Ile Ala Ile Ile Ala Pro Ala Ile Val Pro |
| 152 | Leu Ala Val Ala Ile Val Ala Pro Ala Leu Val Pro |
| 153 | Leu Ala Ile Val Leu Ala Ala Pro Ala Val Leu Pro |
| 154 | Ala Ala Ile Val Leu Ala Leu Pro Ala Val Leu Pro |
| 155 | Ala Leu Leu Val Ala Val Leu Pro Ala Ala Leu Pro |
| 156 | Ala Ala Leu Val Ala Val Leu Pro Val Ala Leu Pro |
| 157 | Ala Ile Leu Ala Val Ala Leu Pro Leu Leu Ala Pro |
| 158 | Ile Val Ala Val Ala Leu Val Pro Ala Leu Ala Pro |
| 159 | Ile Val Ala Val Ala Leu Leu Pro Ala Leu Ala Pro |
| 160 | Ile Val Ala Val Ala Leu Leu Pro Ala Val Ala Pro |
| 161 | Ile Val Ala Leu Ala Val Leu Pro Ala Val Ala Pro |
| 162 | Val Ala Val Leu Ala Val Leu Pro Ala Leu Ala Pro |
| 163 | Ile Ala Val Leu Ala Val Ala Pro Ala Val Leu Pro |
| 164 | Leu Ala Val Ala Ile Ile Ala Pro Ala Val Ala Pro |
| 165 | Val Ala Leu Ala Ile Ala Leu Pro Ala Val Leu Pro |
| 166 | Ala Ile Ala Ile Ala Leu Val Pro Val Ala Leu Pro |
| 167 | Ala Ala Val Val Ile Val Ala Pro Val Ala Leu Pro |
| 168 | Val Ala Ile Ile Val Val Ala Pro Ala Leu Ala Pro |
| 169 | Val Ala Leu Leu Ala Ile Ala Pro Ala Leu Ala Pro |
| 170 | Val Ala Val Leu Ile Ala Val Pro Ala Leu Ala Pro |
| 171 | Ala Val Ala Leu Ala Val Leu Pro Ala Val Val Pro |
| 172 | Ala Val Ala Leu Ala Val Val Pro Ala Val Leu Pro |
| 173 | Ile Val Val Ile Ala Val Ala Pro Ala Val Ala Pro |
| 174 | Ile Val Val Ala Ala Val Val Pro Ala Leu Ala Pro |
| 175 | Ile Val Ala Leu Val Pro Ala Val Ala Ile Ala Pro |
| 176 | Val Ala Ala Leu Pro Ala Val Ala Leu Val Val Pro |
| 177 | Leu Val Ala Ile Ala Pro Leu Ala Val Leu Ala Pro |
| 178 | Ala Val Ala Leu Val Pro Val Ile Val Ala Ala Pro |
| 179 | Ala Ile Ala Val Ala Ile Ala Pro Val Ala Leu Pro |
| 180 | Ala Ile Ala Leu Ala Val Pro Val Leu Ala Leu Pro |
| 181 | Leu Val Leu Ile Ala Ala Ala Pro Ile Ala Leu Pro |
| 182 | Leu Val Ala Leu Ala Val Pro Ala Ala Val Leu Pro |
| 183 | Ala Val Ala Leu Ala Val Pro Ala Leu Val Leu Pro |
| 184 | Leu Val Val Leu Ala Ala Ala Pro Leu Ala Val Pro |
| 185 | Leu Ile Val Leu Ala Ala Pro Ala Leu Ala Ala Pro |
| 186 | Val Ile Val Leu Ala Ala Pro Ala Leu Ala Ala Pro |
| 187 | Ala Val Val Leu Ala Val Pro Ala Leu Ala Val Pro |
| 188 | Leu Ile Ile Val Ala Ala Ala Pro Ala Val Ala Pro |
| 189 | Ile Val Ala Val Ile Val Ala Pro Ala Val Ala Pro |
| 190 | Leu Val Ala Leu Ala Ala Pro Ile Ile Ala Val Pro |
| 191 | Ile Ala Ala Val Leu Ala Ala Pro Ala Leu Val Pro |
| 192 | Ile Ala Leu Leu Ala Ala Pro Ile Ile Ala Val Pro |
| 193 | Ala Ala Leu Ala Leu Val Ala Pro Val Ile Val Pro |
| 194 | Ile Ala Leu Val Ala Ala Pro Val Ala Leu Val Pro |
| 195 | Ile Ile Val Ala Val Ala Pro Ala Ala Ile Val Pro |
| 196 | Ala Val Ala Ala Ile Val Pro Val Ile Val Ala Pro |
| 197 | Ala Val Leu Val Leu Val Ala Pro Ala Ala Ala Pro |
| 198 | Val Val Ala Leu Leu Ala Pro Leu Ile Ala Ala Pro |
| 199 | Ala Ala Val Val Ile Ala Pro Leu Leu Ala Val Pro |
| 200 | Ile Ala Val Ala Val Ala Ala Pro Leu Leu Val Pro |
| 201 | Leu Val Ala Ile Val Val Leu Pro Ala Val Ala Pro |
| 202 | Ala Val Ala Ile Val Val Leu Pro Ala Val Ala Pro |
| 203 | Ala Val Ile Leu Leu Ala Pro Leu Ile Ala Ala Pro |
| 204 | Leu Val Ile Ala Leu Ala Ala Pro Val Ala Leu Pro |
| 205 | Val Leu Ala Val Val Leu Pro Ala Val Ala Leu Pro |
| 206 | Val Leu Ala Val Ala Ala Pro Ala Val Leu Leu Pro |
| 207 | Ala Ala Val Val Leu Leu Pro Ile Ile Ala Ala Pro |
| 208 | Ala Leu Leu Val Ile Ala Pro Ala Ile Ala Val Pro |
| 209 | Ala Val Leu Val Ile Ala Val Pro Ala Ile Ala Pro |
| 210 | Ala Leu Leu Val Val Ile Ala Pro Leu Ala Ala Pro |
| 211 | Val Leu Val Ala Ala Ile Leu Pro Ala Ala Ile Pro |
| 212 | Val Leu Val Ala Ala Val Leu Pro Ile Ala Ala Pro |
| 213 | Val Leu Ala Ala Ala Val Leu Pro Leu Val Val Pro |
| 214 | Ala Ile Ala Ile Val Val Pro Ala Val Ala Val Pro |
| 215 | Val Ala Ile Ile Ala Val Pro Ala Val Val Ala Pro |
| 216 | Ile Val Ala Leu Val Ala Pro Ala Ala Val Val Pro |
| 217 | Ala Ala Ile Val Leu Leu Pro Ala Val Val Val Pro |
| 218 | Ala Ala Leu Ile Val Val Pro Ala Val Ala Val Pro |
| 219 | Ala Ile Ala Leu Val Val Pro Ala Val Ala Val Pro |
| 220 | Leu Ala Ile Val Pro Ala Ala Ile Ala Ala Leu Pro |
| 221 | Leu Val Ala Ile Ala Pro Ala Val Ala Val Leu Pro |
| 222 | Val Leu Ala Val Ala Pro Ala Val Ala Val Leu Pro |
| 223 | Ile Leu Ala Val Val Ala Ile Pro Ala Ala Ala Pro |
| 224 | Ile Leu Val Ala Ala Ala Pro Ile Ala Ala Leu Pro |
| 225 | Ile Leu Ala Val Ala Ala Ile Pro Ala Ala Leu Pro |
| 226 | Val Ile Ala Ile Pro Ala Ile Leu Ala Ala Ala Pro |
| 227 | Ala Ile Ile Ile Val Val Pro Ala Ile Ala Ala Pro |
| 228 | Ala Ile Leu Ile Val Val Ala Pro Ile Ala Ala Pro |
| 229 | Ala Val Ile Val Pro Val Ala Ile Ile Ala Ala Pro |
| 230 | Ala Val Val Ile Ala Leu Pro Ala Val Val Ala Pro |
| 231 | Ala Leu Val Ala Val Ile Ala Pro Val Val Ala Pro |
| 232 | Ala Leu Val Ala Val Leu Pro Ala Val Ala Val Pro |
| 233 | Ala Leu Val Ala Pro Leu Leu Ala Val Ala Val Pro |
| 234 | Ala Val Leu Ala Val Val Ala Pro Val Val Ala Pro |
| 235 | Ala Val Ile Ala Val Ala Pro Leu Val Val Ala Pro |
| 236 | Ala Val Ile Ala Leu Ala Pro Val Val Val Ala Pro |
| 237 | Val Ala Ile Ala Leu Ala Pro Val Val Val Ala Pro |
| 238 | Val Ala Leu Ala Leu Ala Pro Val Val Val Ala Pro |
| 239 | Val Ala Ala Leu Leu Pro Ala Val Val Val Ala Pro |
| 240 | Val Ala Leu Ala Leu Pro Ala Val Val Val Ala Pro |
| 241 | Val Ala Leu Leu Ala Pro Ala Val Val Val Ala Pro |

1. Solubilization Domain

The iCP (improved cell-permeable)—mParkin recombinant protein provided according to the present disclosure may further comprise at least one solubilization domain in addition to a modified Parkin and an aMTD domain. In one embodiment, the iCP (improved cell-permeable)—mParkin recombinant protein may comprise a modified Parkin, an aMTD domain, and a solubilization domain. In another embodiment, the iCP (improved cell-permeable)—mParkin recombinant protein may comprise modified Parkin, an aMTD domain, and a solubilization domain.

Given a solubilization domain, the iCP (improved cell-permeable)—mParkin recombinant protein of the present disclosure enjoys the advantage of improving in solubility. In one embodiment, the solubilization domain includes a peptide that acts to increase solubility of a bioactive molecule. In an exemplary embodiment, the solubilization domain may include the amino acid sequence of MAEQSDKDVKYYTLEEIQKHKDSKSTWLILHHKVYDLTKFLEEHPGGEEVLGEQAGGDAT ENFEDVGHSTDARELSKTYIIGELHPDDRSKIAKPSETL (SEQ ID No: 242). However, the solubilization domain is not limited thereto and may be any domain that is known to increase solubility of a bioactive molecule.

II. Specific Example of iCP (Improved Cell-Permeable)—mParkin Recombinant Protein

The iCP (improved cell-permeable)—mParkin recombinant protein provided according to one embodiment of the present disclosure can be represented by a structural formula selected from among A-B, B-A, A-B-C, A-C-B, B-A-C, B-C-A, CA-B, C-B-A, and A-C-B-C. In the structural formulas, A is accounted for by an aMTD domain, B by a modified Parkin, and C by a solubilization domain.

In an exemplary embodiment, the iCP (improved cell-permeable)—mParkin recombinant protein provided according to the present disclosure may include the following amino acid sequence. However, the amino acid sequence of the iCP (improved cell-permeable)—mParkin recombinant protein is not limited thereto, but may be any sequence that is possible from the combinations described in section I. iCP (improved cell-permeable)—mParkin recombinant protein.

TABLE 2
Specific Example of iCP(improved cell-permeable) -
mParkin recombinant protein

| | | SEQ ID |
|---|---|---|
| No. | Sequence | No. |
| iCP- | AVALIVVPALAPQEMNATGGDDPRNAAGGCEREPQSLTRV | 243 |
| mParkin | DLSSSVLPGDSVGLAVILHTDSRKDSPPAGSPAGRSIYNSFY | |
| | VYCKGPCQRVQPGKLRVQCSTCRQATLTLTQGPSCWDDVL | |
| | IPNRMSGECQSPHCPGTSAEFFFKCGAHPTSDKETSVALHLI | |
| | ATNSRNITCITCTDVRSPVLVFQCNSRHVICLDCFHLYCVTR | |
| | LNDRQFVHDPQLGYSLPCVAGCPNSLIKELHHFRILGEEQY | |
| | NRYQQYGAEECVLQMGGVLCPRPGCGAGLLPEPDQRKVT | |
| | CEGGNGLGCGFAFCRECKEAYHEGECSAVFEASGTTTQAY | |
| | RVDERAAEQARWEAASKETIKKTTKPCPRCHVPVEKNGGC | |
| | MHMKCPQPQCRLEWCWNCGCEWNRVCMGDHWFDVMAE | |
| | QSDKDVKYYTLEEIQKHKDSKSTWLILHHKVYDLTKFLEEH | |
| | PGGEEVLGEQAGGDATENFEDVGHSTDARELSKTYIIGELH | |
| | PDDRSKIAKPSETL | |

Moreover, the present disclosure provides not only the amino acid sequence of the iCP (improved cell-permeable)—mParkin recombinant protein, but also a polynucleotide encoding the same, a recombinant expression vector carrying the polynucleotide, and a transformant transformed with the recombinant expression vector.

III. Use of iCP (Improved Cell-Permeable)—mParkin Recombinant Protein

1. Pharmaceutical Composition

One embodiment of the present disclosure provides a composition comprising the iCP (improved cell-permeable)—mParkin recombinant protein. Another embodiment of the present disclosure provides a composition comprising the iCP (improved cell-permeable)—mParkin recombinant protein as an active ingredient. In one embodiment, the composition may be a pharmaceutical composition for treatment or prevention of a disease.

The pharmaceutical composition provided according to one embodiment of the present disclosure may further comprise a vehicle. The pharmaceutically acceptable vehicle contained in the pharmaceutical composition of the present disclosure is usually used for formulation. Examples of the vehicle include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxy benzoate, propyl hydroxy benzoate, talc, magnesium stearate, mineral oil, and the like, but are not limited thereto. In addition to the above ingredients, the pharmaceutical composition of the present disclosure may further contain a lubricant, a wetting agent, sweetener, a colorant, a flavorant, an emulsifier, a suspending agent, a preservative, and the like. For details of pharmaceutically acceptable vehicles and suitable formulations, reference may made to Remington's Pharmaceutical Sciences (19th ed., 1995).

The pharmaceutical composition according to the present disclosure may be formulated using at least one diluent or excipient, usually used in the art, such as a filler, an extender, a binder, a wetting agent, a disintegrant, a surfactant, and so on.

In one embodiment, solid formulations for oral administration include tablets, pills, powders, granules, capsules, troches, etc. These solid formulations may be prepared by mixing at least one compound of the present disclosure with one or more excipients, for example, starch, calcium carbonate, sucrose, lactose, gelatin, etc. In addition, a lubricant such as magnesium stearate, talc, etc. is employed in addition to simple excipients. In another embodiment, liquid formulations for oral administration include a suspension, a solution for internal use, an emulsion, a syrup, etc. In addition to water commonly used as a simple diluent and liquid paraffin, various excipients, for example, wetting agents, sweetening agents, flavors, preservatives, etc. may be included. Formulations for parenteral administration include sterilized aqueous solutions, non-aqueous solvents, suspending agents, emulsions, lyophilizates, suppositories, etc. Propylene glycol, polyethylene glycol, vegetable oils such as olive oil, injectable esters such as ethyl oleate, etc. may be used as non-aqueous solvents and suspending agents. Bases for suppositories may include witepsol, macrogol, tween 61, cacao butter, laurin butter, glycerinated gelatin, etc.

The iCP (improved cell-permeable)—mParkin recombinant protein has cell permeability and may act to protect neuronal cells from neurotoxins. In greater detail, the iCP (improved cell-permeable)—mParkin recombinant protein promotes mitophagy in a mitochondria damaged condition and suppresses the accumulation of pathological alpha-synuclein. That is, the iCP (improved cell-permeable)—mParkin recombinant protein plays a role in protecting neuronal cells according to the mechanisms.

Accordingly, the iCP (improved cell-permeable)—mParkin recombinant protein can be contained in a pharmaceutical composition for prevention, treatment, or alleviation of a neuronal cell damage-related disease or can be used as a medicine or for preparing a medicine. In one embodiment, the neuronal cell damage-related disease may include neurodegenerative disease, examples of which include Parkinson's disease, Alzheimer's disease, Huntington's disease, Amyotrophic lateral sclerosis (ALS), and Motor neuron disease, but are not limited thereto. Any disease that is known as neuronal cell damage-related disease may be included.

2. Method of Treating

In one embodiment of the present disclosure, the iCP (improved cell-permeable)—mParkin recombinant protein can be used for treating a disease. More specifically, one embodiment of the present disclosure provides a method for treatment of a disease, the method comprising administering a composition comprising the iCP (improved cell-permeable)—mParkin recombinant protein to a subject in need thereof. In this context, the subject may mean a mammal including humans.

According to intended modalities, the composition provided in one embodiment of the present disclosure may be orally or parenterally administered (for example, intravenously, subcutaneously, intraperitoneally, or topically). Administration doses may be properly determined by a person skilled in the art, depending on patient's state and body weight, the severity of disease, dosage forms of drugs, administration routes and time, etc.

The composition according to the present disclosure is administered in a pharmaceutically effective amount. As used herein, the term “pharmaceutically effective amount” refers to an amount sufficient to treat diseases, at a reasonable benefit/risk ratio applicable to any medical treatment. The effective dosage level may be determined depending on various factors including the type and severity of disease, the activity of drugs, the sensitivity to drugs, the time of administration, the route of administration, excretion rate, the duration of treatment, drugs used in combination with the composition, and other factors known in the medical field. The composition of the present invention may be administered as a sole therapeutic agent or in combination with other therapeutic agents, and may be administered sequentially or simultaneously with conventional therapeutic agents. The composition can be administered in a single or multiple dosage form. It is important to administer the composition in the minimum amount that can exhibit the maximum effect without causing side effects, in view of all the above-described factors, and the amount can be easily determined by a person skilled in the art.

In detail, an effective amount of the compound according to the present disclosure may vary depending on the age, sex, and body weight of the patient. Generally, the compound may be administered in an amount of 0.1 to 100 mg per kg of body weight and preferably in an amount of 0.5 to 10 mg per kg of body weight every day or every other day, or one to three times a day. The dose may be increased or decreased depending on administration route, severity of obesity, sex, body weight, age, etc. and thus does not limit the scope of the present disclosure in any way.

In the context of the method for treatment of a disease, the disease includes all the disclosure of section 2. Pharmaceutical composition on diseases. That is, the present disclosure provides a method for treatment of neurodegenerative disease, the method comprising administering a pharmaceutical composition comprising the iCP (improved cell-permeable)—mParkin recombinant protein to a subject in need thereof. In addition, the present invention provides a method for treatment of neural cell damage-related diseases including Parkinson's disease, Alzheimer's disease, Huntington's disease, Amyotrophic lateral sclerosis (ALS), and Motor neuron disease, the method comprising administering a pharmaceutical composition comprising the iCP (improved cell-permeable)—mParkin recombinant protein to a subject in need thereof.

IV. Preparation Method for iCP (Improved Cell-Permeable)—mParkin Recombinant Protein

One embodiment of the present disclosure provides a preparation method for an iCP (improved cell-permeable)—mParkin recombinant protein. The method may comprise the following steps:

1. Preparing a Recombinant Expression Vector Comprising a Polynucleotide Sequence Encoding an iCP-mParkin Recombinant Protein.

The preparation method may comprise a step of preparing a recombinant expression vector comprising a polynucleotide sequence encoding an iCP-mParkin recombinant protein. In this regard, the iCP-mParkin recombinant protein is accounted for by the disclosure of section I. iCP (improved cell-permeable)—mParkin recombinant protein.

As used herein, the term “recombinant expression vector” refers to a vector for expressing a recombinant peptide or protein. In addition, the vector of the present disclosure may be constructed with a prokaryotic cell or eukaryotic cell serving as a host cell. The recombinant expression vector of the present disclosure may be, for example, a bacteriophage vector, a cosmid vector, a YAC (Yeast Artificial Chromosome) vector, a plasmid, etc. The vectors utilized in the present disclosure can be constructed using various methods known in the art.

2. Preparing a Transformant Using the Recombinant Expression Vector

The preparation method may comprise a step of preparing a transformant using the recombinant expression vector. So long as it is known as a host cell capable of producing a recombinant protein, any host cell may be used for transformation with the recombinant expression vector. Examples of the host cell include bacteria, yeasts, fungi, etc., but are not limited thereto. In one embodiment, an E. coli strain may be used. In an exemplary embodiment, E. coli BL21star (DE3), NEB express strain may be used. In a more exemplary embodiment, an E. coli NEB express strain may be used. With the E. coli NEB express strain, a higher IB (inclusion body) yield can be obtained. However, no limitations are imparted thereto, but Agrobacterium sp. Strains such as Agrobacterium A4, Bacillus sp. strains such as Bacillus subtilis, Pseudomonas sp. strains, and Lactobacillus sp. strains may be used as host cells. The present disclosure is not limited by the examples.

3. Culturing the Transformant

The preparation method may comprise a step of culturing the transformant. In one embodiment, the culturing may include batch fermentation or fed-batch fermentation. In an exemplary embodiment, the culturing may include fed-batch fermentation, which can bring about an improvement in yielding the recombinant protein, compared to batch fermentation.

4. Obtaining the Recombinant Protein Expressed by the Culturing

The preparation method may comprise a step of obtaining the recombinant protein expressed by the culturing. The step of obtaining the recombinant protein may comprise:

1) Washing of an Inclusion Body

The obtaining step may include washing an inclusion body. In this regard, the washing may be conducted once or more times. In one embodiment, two or more rounds of washing may be conducted. In consideration of protein loss, the washing may be simplified to one round. In an exemplary embodiment, the washing includes one-step washing using a washing buffer (pH 8) that contains 5M urea and 50 mM Tris, but with no limitations thereto.

2) Ion Exchange Chromatography

The obtaining step may include performing ion exchange chromatography. In one embodiment, the ion exchange chromatography may be conducted twice. In an exemplary embodiment, of two rounds of ion exchange chromatography, the first ion exchange chromatography may be cation exchange chromatography while the second ion exchange chromatography may be anion exchange chromatography. When two rounds of ion exchange chromatography is performed in the obtaining step, the preparation method for an iCP (improved cell-permeable)—mParkin recombinant protein may omit SEC (Size exclusion chromatography).

In one embodiment, the step of performing the second ion exchange chromatography may include step-elution. The step-elution includes washing in an 8.0 mS/Cm conductivity condition and elution in a 9.0 mS/Cm conductivity condition. The step-elution allows the monomeric iCP-mParkin to be obtained at high yield.

3) Additional Step

The obtaining step may further include additional steps in addition to washing of an inclusion body and performing ion exchange chromatography. In one embodiment, the obtaining step may include denaturation, refolding, and dissolving. In an exemplary embodiment, the obtaining step may include washing of an inclusion body, denaturation, performing the first ion exchange chromatography, performing the second ion exchange chromatography, refolding, and dissolving in that order, but with no limitations thereto. For effectively obtaining a recombinant protein, each step may be modified.

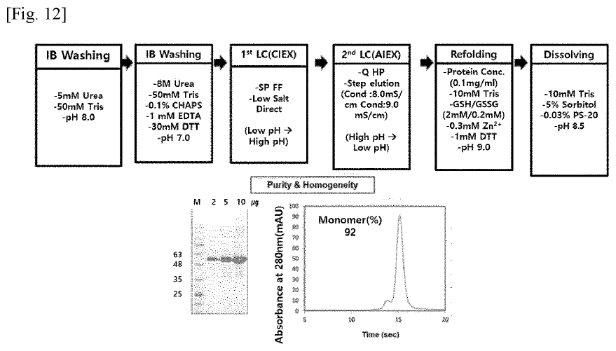

A preferred preparation method for effectively obtaining an iCP (improved cell-permeable)—mParkin recombinant protein is illustrated in FIG. 12.

Hereinafter, the present disclosure will be described in further detail with reference to the following examples. It is to be understood, however, that these examples are for illustrative purposes only and are not intended to limit the scope of the present invention.

### Examples

1. Development of Improved Cell-Permeable Modified Parkin (iCP-mParkin) and Technical Meaning

(1) Structural Modification and Screening and Development of iCP-mParkin to Improve Homogeneity & Stability

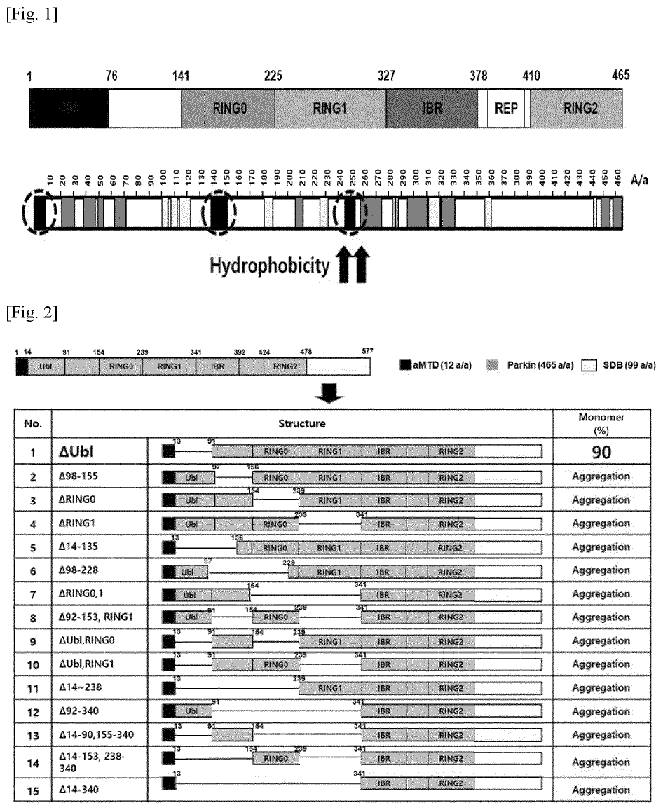

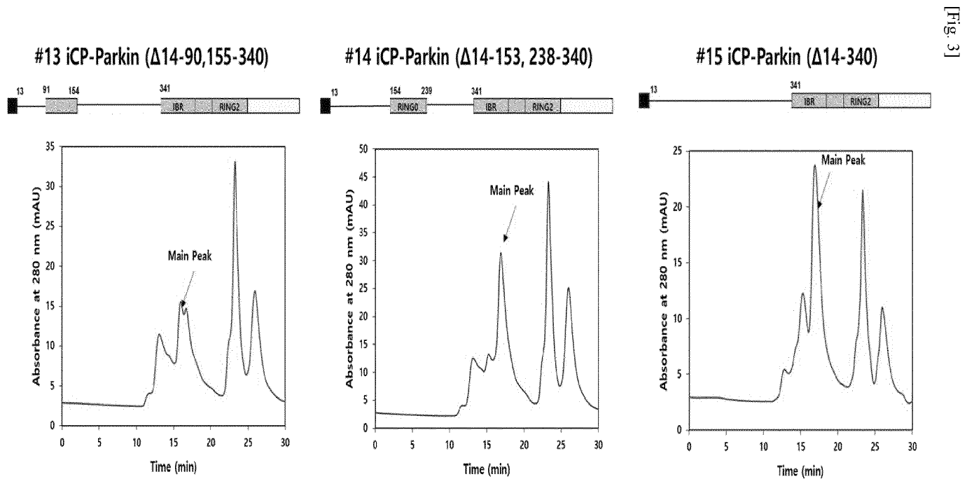

Parkin protein is composed of several domains including N-terminal ubiquitin-like (Ubl), really interesting new gene (RING) 0, 1, 2, in between ring (IBR), and repressor element (REP) domains. RING domains include zinc binding sites, which are important for Parkin structure formation. RING1 domain is E2-Ub binding site and RING2 domain is a main activity site as E3 ubiquitin ligase, which contains critical spot for enzymatic activity of Parkin domain (FIG. 1). Furthermore, the hydrophobicity-dense region can cause aggregation and instability of Parkin protein due to its hydrophobic interaction (FIG. 1). In order to produce a higher stable Parkin structure based on this rationale including structure-activity-relationship (SAR) and hydrophobic density, the clones to express the varied structures of modified iCP-Parkin proteins were developed and the induction and purification of the corresponding proteins were investigated (FIG. 2).

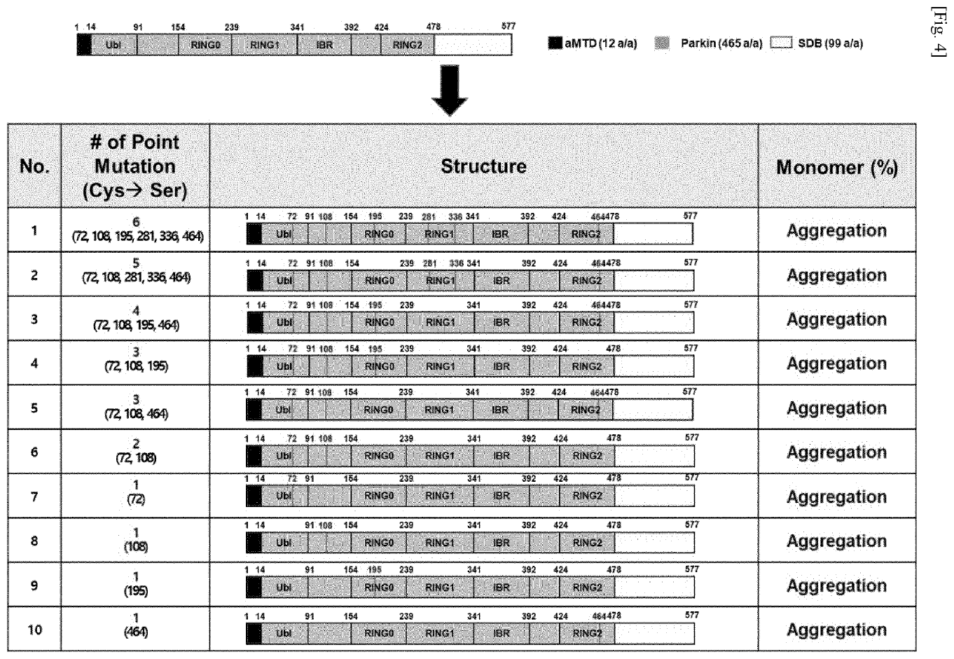

Most of iCP-Parkin variants were aggregated during refolding steps and could not be analyzed (FIG. 2), and some variants (No. 13-15) showed heterogeneous species as analyzed after purification by HPLC analysis (FIG. 3).

Cysteine residues existing in native Parkin sequence were altered to serine in order to prevent the formation of intra and inter-molecular disulfide bond hence increasing structural stability (FIG. 4).

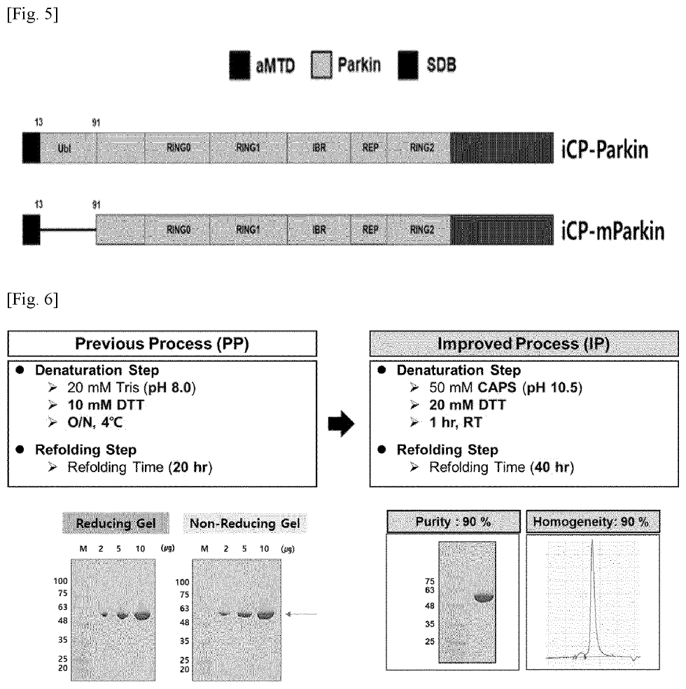

Total of 25 variant structures were designed and screened, where only 1 structure showed superior stability. Rest of 24 showed severe aggregation and purification process had to halt before column purification work. The UBL domain-deleted structure from the native Parkin [aMTD524- Parkin (ΔUBL)-SDB] didn't show aggregation (FIG. 5). This modified structure was named improved cell-permeable modified Parkin (iCP-mParkin) and was used for following experiments. In summary, the variant deleted Ubl domain only could be purified with high stability as an advanced iCP-Parkin whose therapeutic potential was already demonstrated in prior studies by using PD cell & animal models.

The sequence of iCP-mParkin is as follows:

(SEQ ID No: 243)
AVALIVVPALAPQEMNATGGDDPRNAAGGCEREPQSLTRVDLSSSVLP
GDSVGLAVILHTDSRKDSPPAGSPAGRSIYNSFYVYCKGPCQRVQPGK
LRVQCSTCRQATLTLTQGPSCWDDVLIPNRMSGECQSPHCPGTSAEFF
FKCGAHPTSDKETSVALHLIATNSRNITCITCTDVRSPVLVFQCNSRH
VICLDCFHLYCVTRLNDRQFVHDPQLGYSLPCVAGCPNSLIKELHHFR
ILGEEQYNRYQQYGAEECVLQMGGVLCPRPGCGAGLLPEPDQRKVTCE
GGNGLGCGFAFCRECKEAYHEGECSAVFEASGTTTQAYRVDERAAEQA
RWEAASKETIKKTTKPCPRCHVPVEKNGGCMHMKCPQPQCRLEWCWNC
GCEWNRVCMGDHWFDVMAEQSDKDVKYYTLEEIQKHKDSKSTWLILHH
KVYDLTKFLEEHPGGEEVLGEQAGGDATENFEDVGHSTDARELSKTYI
IGELHPDDRSKIAKPSETL

(2) Optimization of Purification Step for Massive Production

To deal with this issue, the Improved Process (IP) was developed after re-optimization by varying chemicals and physical conditions in denaturation and refolding in E. coli system, capable of manufacturing monomeric recombinant protein with high purity (FIG. 6).

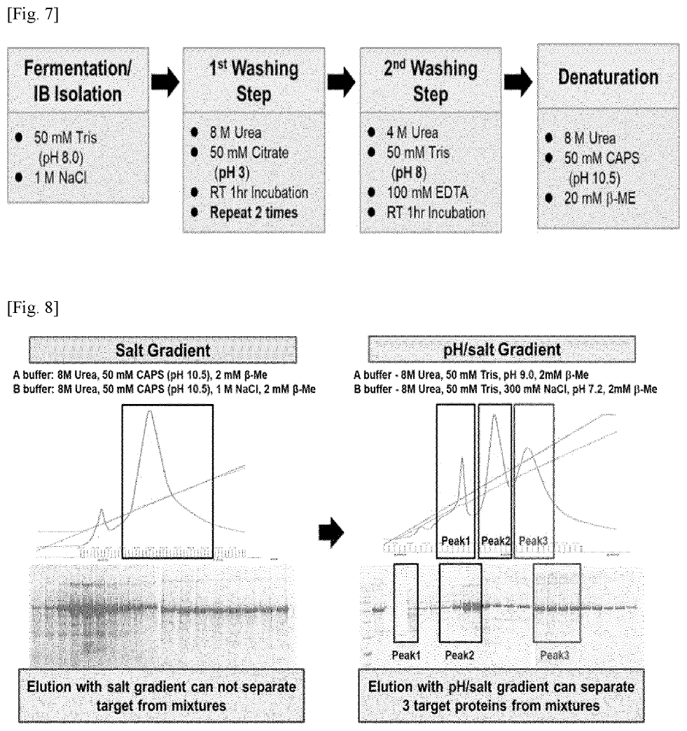

Process condition screening was focused on a wide range of procedures including inclusion body (IB) washing, column screening and elution method screening. pH conditions and buffering agents were modified for denaturation and refolding processes. As opposed to the previous process where refolding was carried out following HIC purification, HIC was replaced by AIEX and was performed after the refolding process. Refolding protein concentration was corrected to 0.1 mg/ml. SEC purification was added following AIEX. IB washing additionally performed prior to denaturation, could enhance the purity of recombinant proteins (FIG. 7). IB washing process consists of two steps: 1) 1st wash with lower pH buffer, 2) 2^{nd} wash with lower urea concentration and higher pH buffer. This process allowed reduction of impurities (FIGS. 7 and 8).

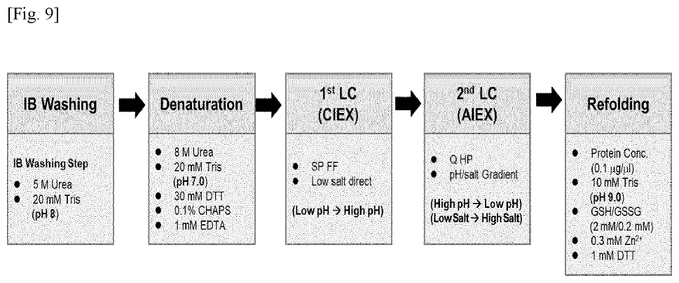

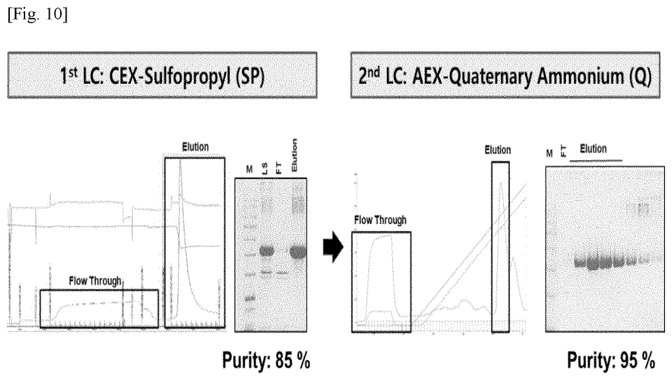

Additional process development was required from the improved process (IP) in prior to large-scale production. The two-step column purifications of AIEX and SEC in the improved process (IP) was replaced with CIEX and AIEX, and gradient elution was replaced by step-elution in order to simplify the elution process in consideration of the larger working volume of large-scale production. Two-step IB washing was also simplified to one step washing process using pH 8 washing buffer, which reduced the loss of target protein while effectively removing impurities (FIGS. 9 and 10). Monomeric iCP-mParkin was purified by varied elution methods as follows: direct elution from low to high pH At CIEX, and gradient elution from high to low pH and low to high NaCl At AIEX (FIGS. 9 and 10).

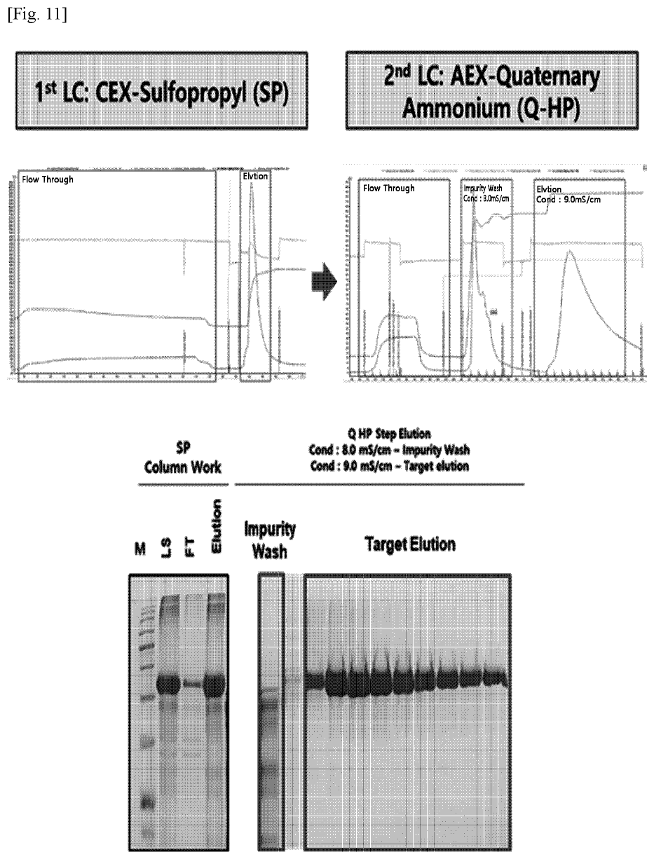

Furthermore, as a further modification of the elution method, the gradient-elution way was replaced into the step-elution. In detail, in prior to the elution step of 2^{nd} LC, a washing step was added using 8.0 mS/Cm conductivity condition to wash out impurities. Conductivity of 9.0 mS/Cm was used for target elution which gave higher yield of monomeric iCP-mParkin (FIG. 11).

Therefore, such modified process compatible with large-scale production was developed as named Final Process (FP). With FP developed based on above-described optimization, iCP-mParkin with higher than 90% (˜92%) homogeneity/purity could be manufactured without SEC column purification (FIG. 12).

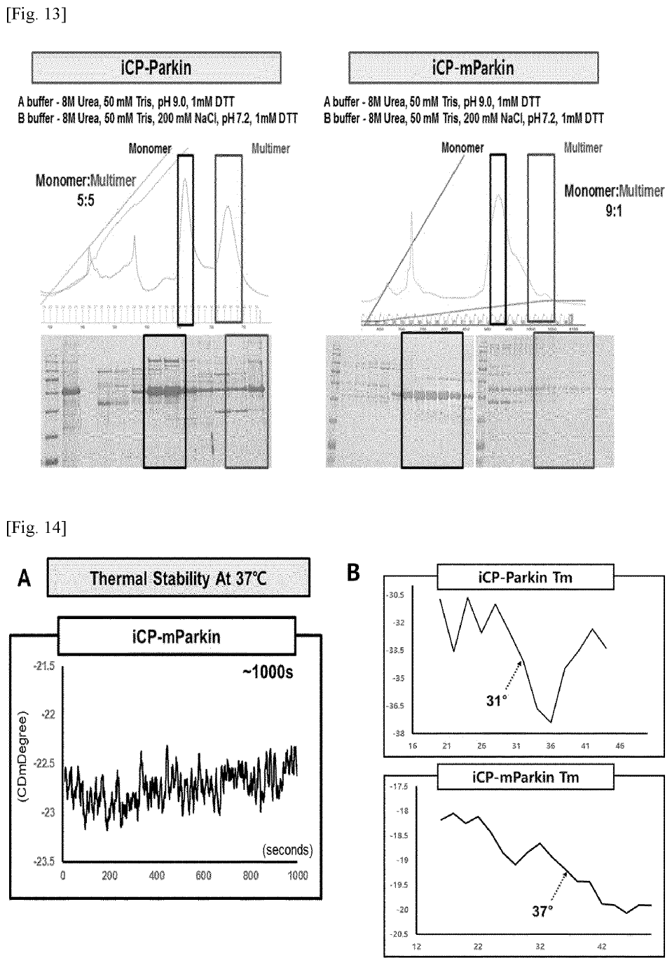

In comparison, iCP-mParkin showed significantly higher monomer portion (FIG. 13).

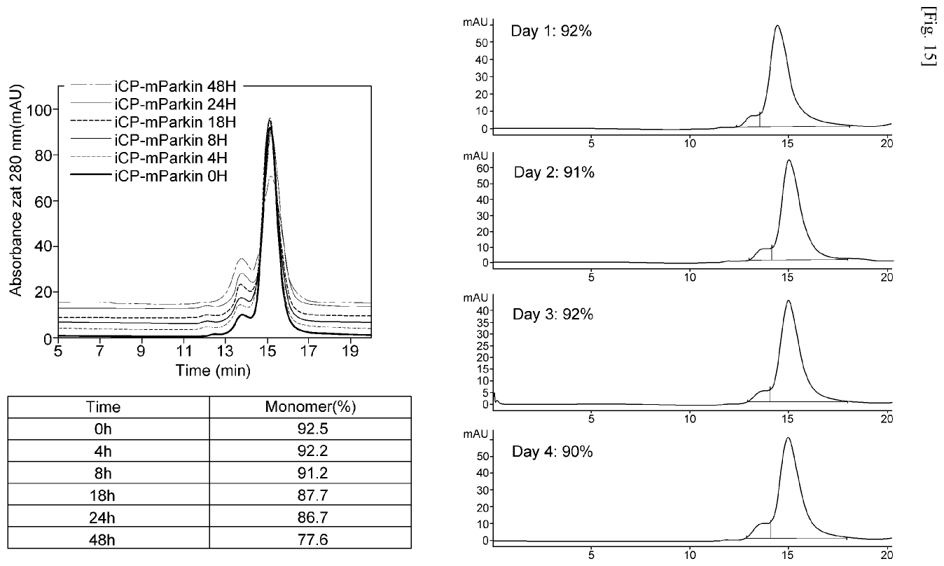

A circular dynamic (CD) analysis was performed to verify the improved structural stability of iCP-mParkin at 37° C. Compared to iCP-Parkin (˜600 seconds), iCP-mParkin show thermal stability, confirming that the structure was maintained during the measurement for 1,000 seconds (FIG. 14A). This was a 67% increase over iCP-mParkin's 37° C. stability. Since the stability after 1000 seconds has not been measured, it is believed that there will be more stability and will be updated later. Tm of iCP-mParkin was measured to be 6° C. higher than that of iCP-Parkin (FIG. 14B). Extended experiments were conducted to further demonstrate the superior stability of iCP-mParkin.

iCP-mParkin is shown to be more stable at 37° C. compared to iCP-Parkin (FIG. 15). iCP-mParkin displayed less than 5% of its monomeric portion loss after 24 hours at 37° C., whereas iCP-Parkin lost over 15%. This indicates better suitability of iCP-mParkin as a clinical therapeutic agent in consideration of the necessity of a therapeutic agent to remain stable in the body until manifesting clinical effect.

When purified using FP, iCP-mParkin showed 92% homogeneity/purity via HPLC analysis. In addition, the two structures showed even greater differences in stability when stored under room temperature of 25° C. Following 4 days of storage, iCP-mParkin showed only 2% loss of monomer purity (FIG. 15). Therefore, HPLC analysis was performed to measure iCP-mParkin's ability to maintain monomer at 37 and 25° C. After checking that no aggregation occurred for 8 hours at 37° C., it is considered to be stable at body temperature for 8 hours, and at room temperature (25° C.), it is believed that iCP-mParkin can be stabilized for 4 days and used as a protein drug.

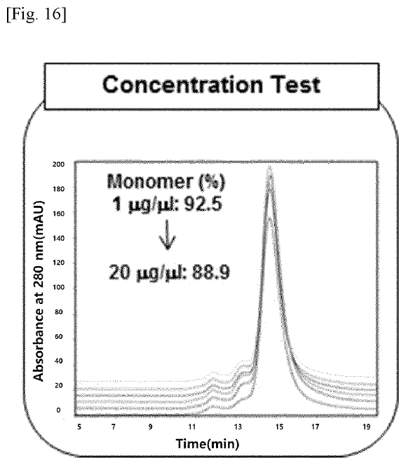

When the stability was assessed at higher protein concentrations (10 ug/ul), iCP-mParkin remained stable even at 20 ug/ul compared to iCP-Parkin with an increased higher multimer portions (FIG. 16). This is an excellent stability compared to the rapid drop of yield (when iCP-Parkin is concentrated (more than 10 ug/ul).

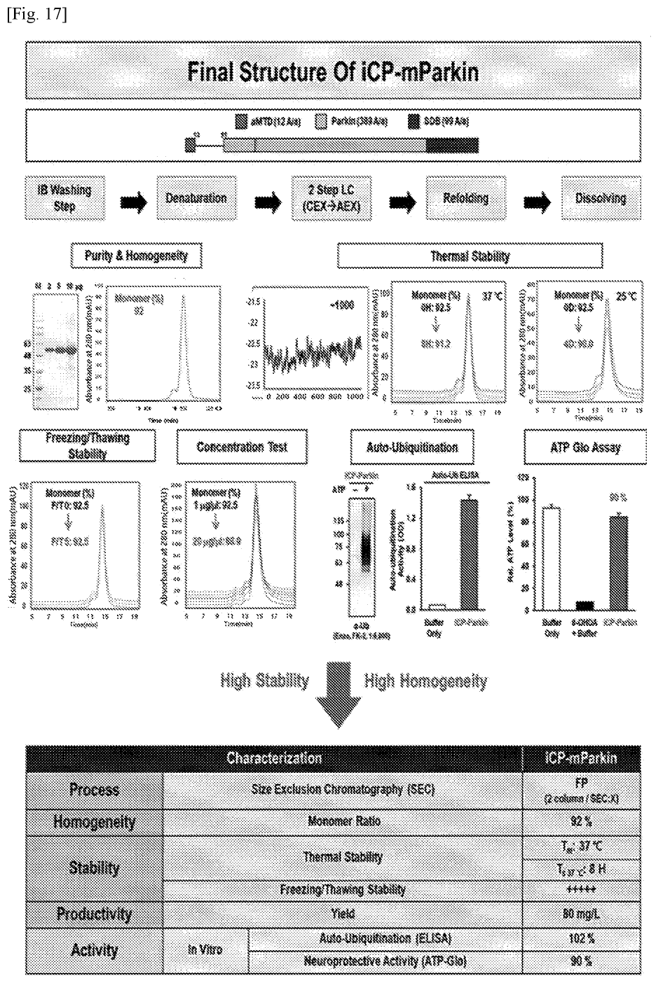

In conclusion, with process development and structure modification, iCP-mParkin can solve several previous limitation and issues (e.g., low stability & monomer yield with heterogeneity, and high-cost SEC usage) of the prior art iCP-Parkin, thus leading to be developed as an advanced drug material with a benefit of a powerful therapeutic potential of iCP-Parkin, which is already proven in peer-review journal publication. iCP-mParkin, a modified structure of iCP-Parkin with the Ubl domain deleted, was selected as the final structure of iCP-Parkin based on its superior purity, homogeneity, stability, and biological activity when produced with the FP as follows (FIG. 17):
Structure Optimization: UBL deletion (thermal stability, 31° C.→37° C.)
Process Optimization (1): increased homogeneity (monomer yield, 81%→92%) by modifying the denaturation/refolding conditions.
Process Optimization (2): removed impurity by re-optimizing IB washing/elution conditions.
Process Optimization (3): replaced size exclusion chromatography (SEC) column.
Process Optimization (4): 20-fold increased protein yield throughout fed-batch fermentation for mass production (4→80 mg/L).

(3) Fed-Batch Fermentation for Increasing Cell Mass

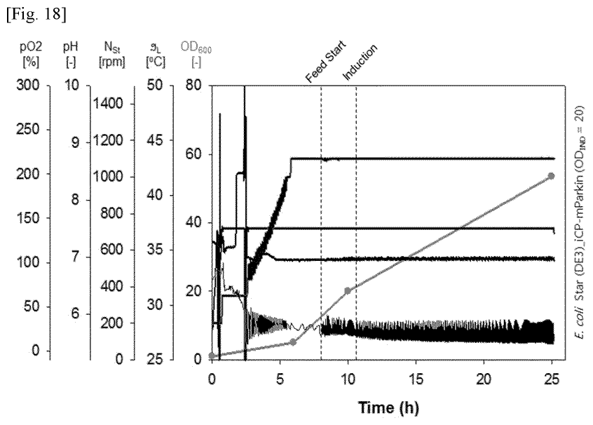

Newly developed iCP-mParkin was structurally stable and therapeutically functional but lacked protein yield. In order to increase final yield of the protein, fed-batch fermentation was used to replace batch fermentation. Unlike batch fermentation, fed-batch fermentation uses continuous supply of nutrients to maximize cell mass and protein yield, which led to approximately 10-fold increase in cell mass harvest to be used for subsequent purification work (FIG. 18).

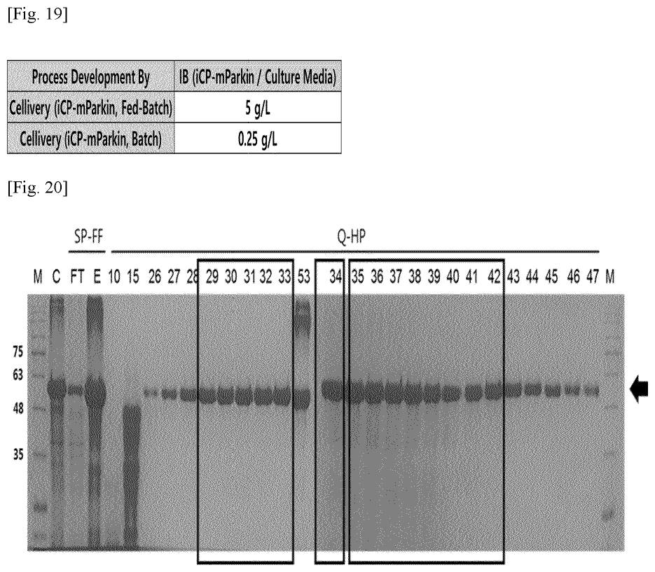

Fed-batch fermentation demonstrated approximately 20-fold increase in yield in comparison to that of Cellivery's previous fermentation process. This yield and process was considered sufficient for tech-transfer to a cGMP-certified CMO (FIG. 19). Furthermore, as E. coli cultivation method, the fed-batch fermentation was optimized to produce iCP-mParkin with high yield in GMP level CMO for drug development. Cell mass was increased 20-fold by this method.

iCP-mParkin products from the novel Fed-batch fermentation and previous batch fermentation are identical (FIG. 20).

(4) Production iCP-mParkin at CMO for Large-Scale Production

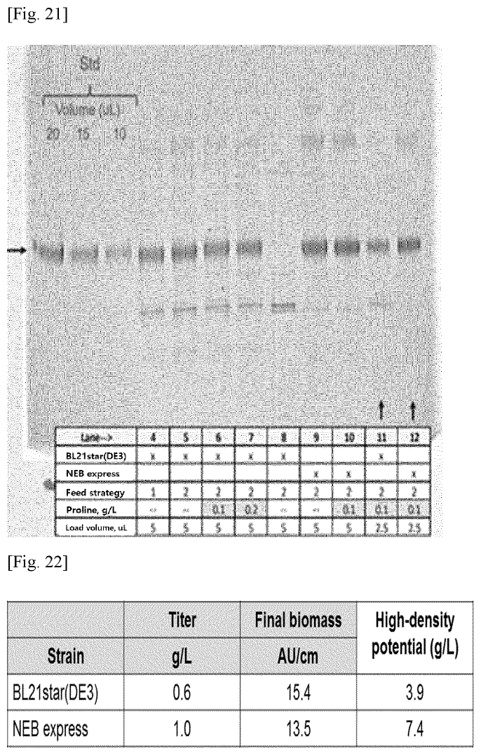

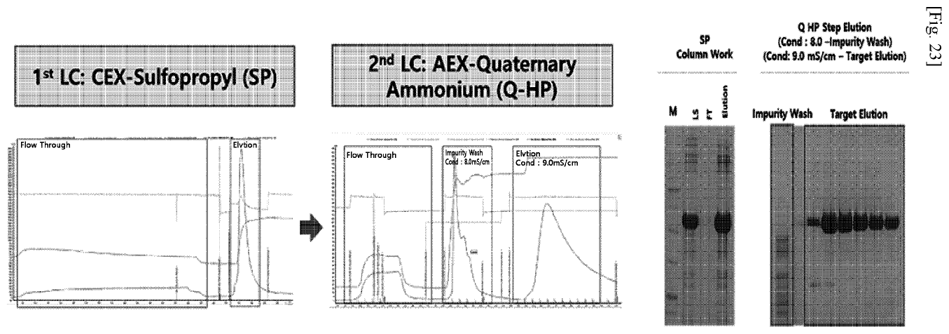

FP developed by Cellivery was tech-transferred to a global cGMP-level CDMO. E. coli cell line development was completed to produce iCP-mParkin at CMO (FIGS. 21 and 22). E. coli cell lines (16 types) made by combining 10 kinds of host strain and 5 types of plasmids, were screened by cloning and transformation. Throughout high-throughput screening, the optimal condition and cell line was chosen by comparing the expression level and purity depending on vectors, induction system (e.g., IPTG, Rhamnose), and additives (e.g., proline/glucose) as well as the cultivation condition. As a result, NEB express showed the highest productivity (purity and expression yield) of iCP-mParkin (FIG. 21).

The high-density potential of IB yield in NEB express stain system is expected as 7.4 g/L, which is much higher than BL21 star (DE3) stain by full scale fed-batch fermentation (FIG. 22).

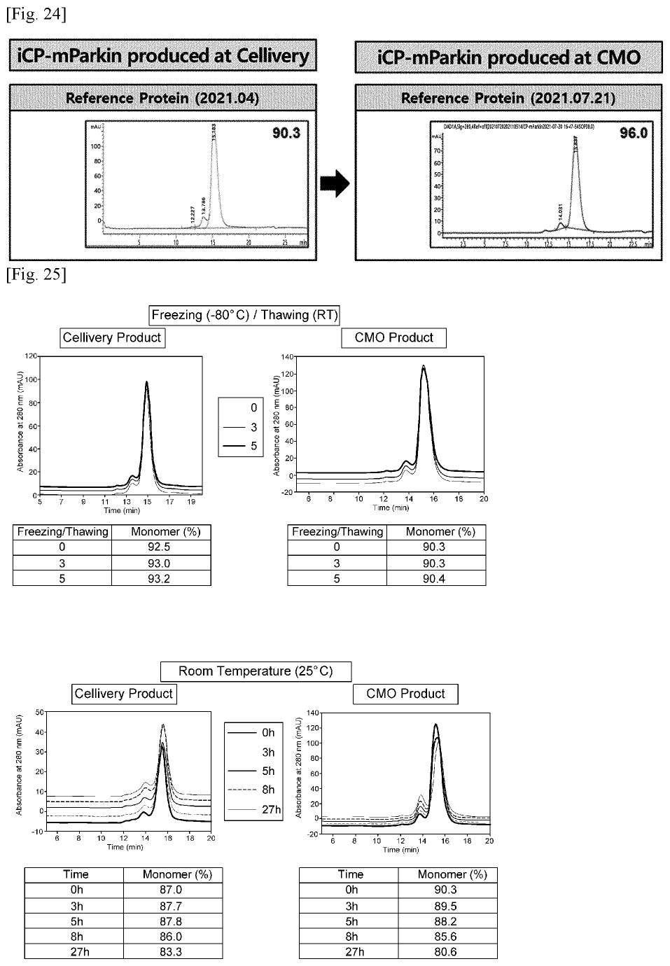

By using these this cell line, iCP-mParkin was produced at 15 L scale. For confirmation of place-to-place variation in protein quality, inhouse production of iCP-mParkin by using CMO cell mass (NEB Express) and FP process showed comparable purity and homogeneity to that produced at CMO (FIGS. 23 and 24).

After purification, the protein properties (e.g., purity, homogeneity, and Stability) and activities are by SDS-PAGE, HPLC. In terms of stability, repeated freezing/thawing and thermal stability of iCP-mParkin produced at inhouse Cellivery and CMO was examined (FIG. 25). As a result, iCP-mParkin maintained structural integrity and stability against physical stress, indicating iCP-mParkin produced by FP at a CMO could be manufactured at a large-scale for clinical drug development.

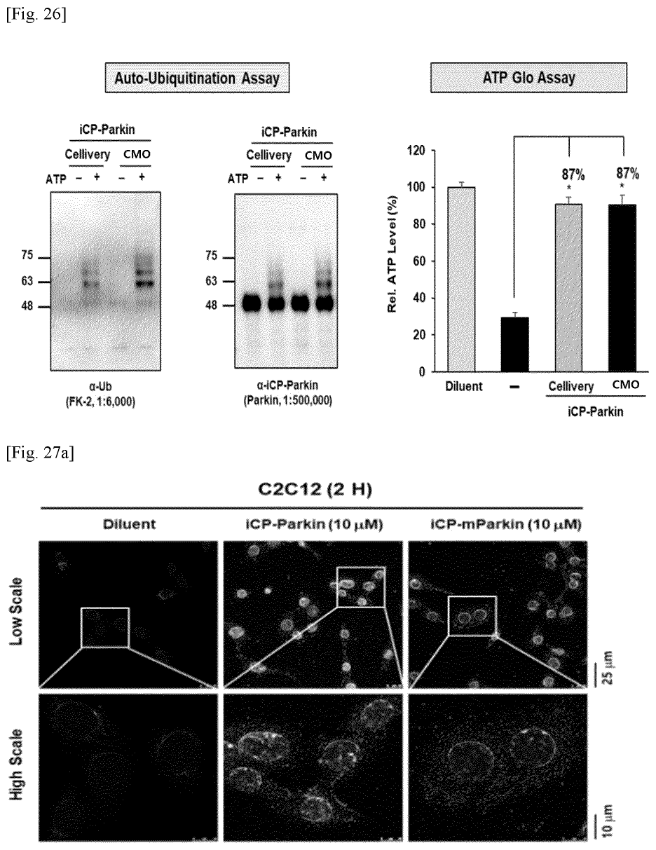

Furthermore, iCP-mParkin produced at CMO has similar great biological activities compared to iCP-mParkin produced at Cellivery (FIG. 26).

2. The Effect of Invention

2-1. Efficacy of iCP-mParkin in Parkinson's Disease (PD) Model

(1) Cell-Permeability of Parkin Recombinant Proteins

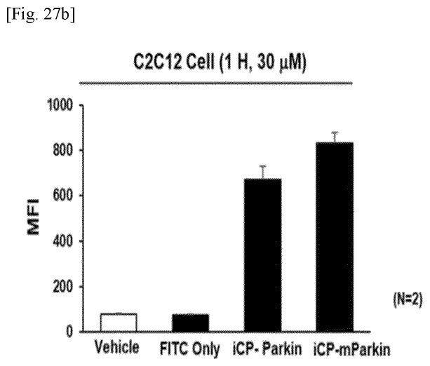

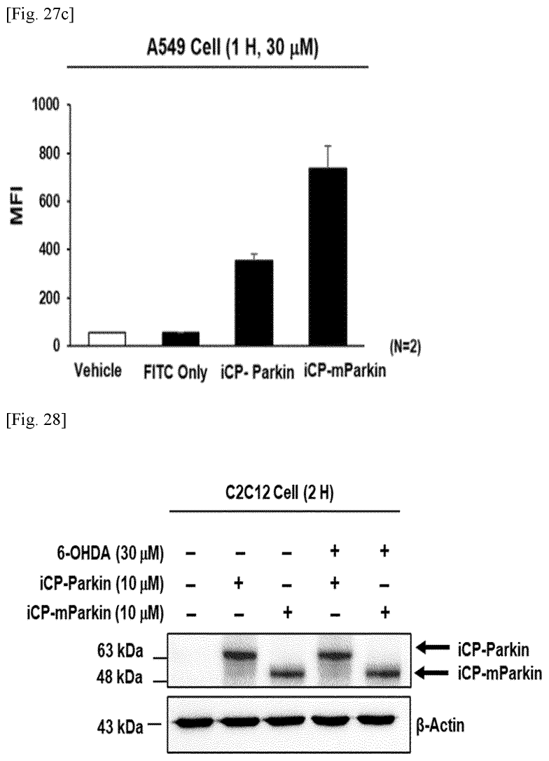

Previously, it has been proved that iCP-Parkin is cell-permeable. Since iCP-mParkin has been generated by structural modification and improved purification process, we investigated the cell-permeability of new parkin recombinant protein compared to previous parkin recombinant protein. Cell permeability of Parkin recombinant proteins was evaluated in C2C12 cells after 2 hour of protein treatment. FITC-labeled the aMTD-bearing Parkin recombinant proteins, iCP-Parkin and iCP-mParkin showed similar cell permeability using fluorescence confocal laser scanning microscopy to monitor protein intracellular localization (FIG. 27A). Next, to quantify the cell-permeability, flow cytometry analysis was performed in C2C12 cells and A549 cell after 1 hours incubation of FITC. Cell permeability of iCP-mParkin is slightly better than iCP-Parkin (FIGS. 27, B and C). These results showed that the iCP-mParkin is successfully able to penetrate into the cells within short time and has improved cell-permeability compared to iCP-Parkin.

To examine the cell-permeability in damaged condition, cells were treated with 6-OHDA mimicking Parkinson's disease, then incubated for 2 hours with iCP-Parkin and iCP-mParkin. The cell-permeability of iCP-Parkin and iCP-mParkin was analysed by western blot analysis. Both of iCP-Parkin and iCP-mParkin were able to penetrate into cells in normal and damaged condition (FIG. 28). This result showed that iCP-mParkin is similar cell-permeability compared to iCP-Parkin.

Delivery of Parkin recombinant proteins. For a visualization of cell-permeability, the Parkin recombinant proteins were conjugated to fluorescein isothiocyanate (FITC) according to the manufacturer's instructions (Sigma-Aldrich, St. Louis, Mo., USA). C2C12 cells were cultured for 24 hours on a coverslip in 24-wells chamber slides, treated with 10 μM of vehicle (culture medium, DMEM), FITC only, FITC-conjugated recombinant proteins for 2 hours at 37° C., and washed three times with cold PBS. Treated cells were fixed in 4% paraformaldehyde (PFA, Junsei, Tokyo, Japan) for 10 minutes at room temperature, washed three times with PBS, and mounted with Mounting Medium (Vector laboratories, Burlingame, Calif., USA) with DAPI (4′,6-diamidino-2-phenylindole) for nuclear staining. The intracellular localization of the fluorescent signal was determined by confocal laser scanning microscopy.

For quantitative cell-permeability, C2C12 cells were treated with 10 μM FITC-labeled recombinant proteins for 1 hour at 37° C., washed three times with cold PBS, treated with proteinase K (5 μg/ml) for 10 min at 37° C. to remove cell-surface bound proteins. Cell-permeability of these recombinant proteins were analyzed by flow cytometry (FACS Calibur; BD, Franklin Lakes, N.J., USA) using the FlowJo analysis software.

For a western blot analysis of cell-permeability, C2C12 cells were treated with 6-OHDA (30 μM), iCP-Parkin (10 μM) and iCP-mParkin (10 μM) for 2 hours. After incubation, cells were lysed and analyzed by western blot analysis.

(2) Biological Activity of Parkin Recombinant Proteins

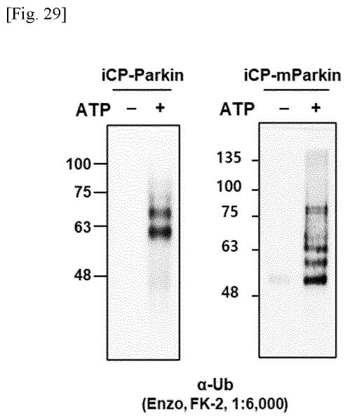

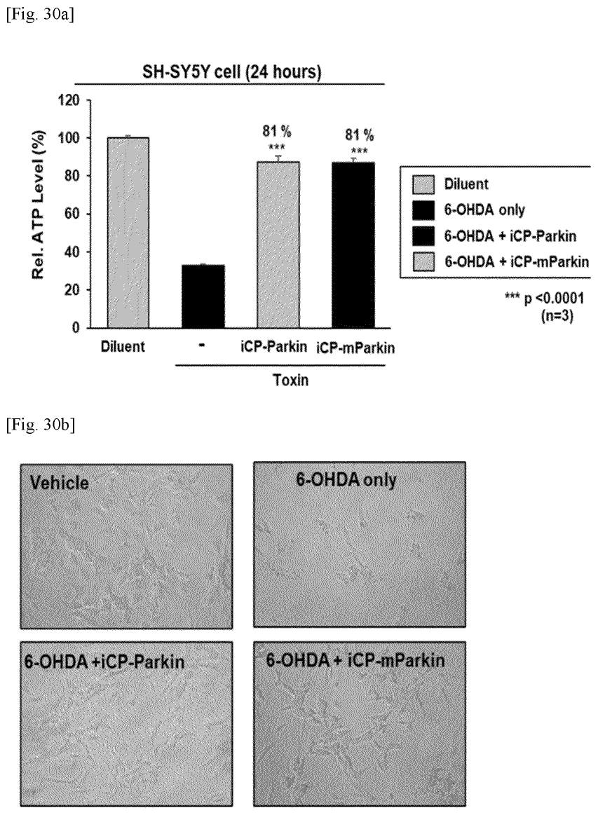

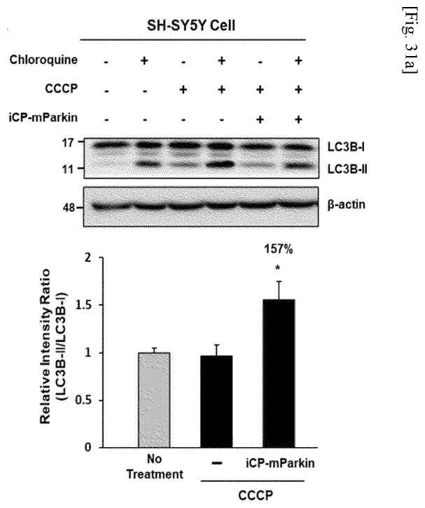

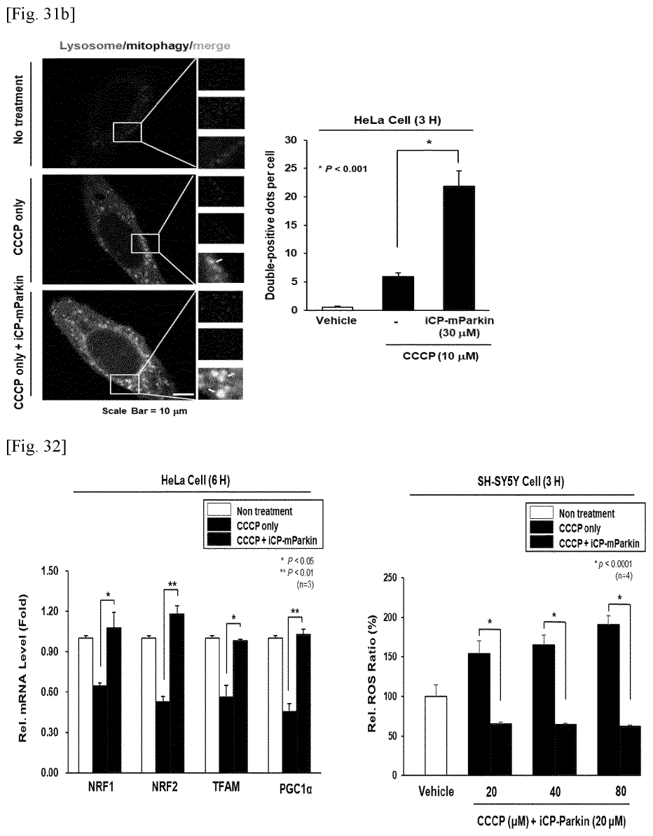

iCP-mParkin shows equivalent auto-ubiquitination activity as E3 ubiquitin ligase and cytoprotective activity in ATP Glo assay (FIGS. 29 and 30). In addition, iCP-mParkin showed equivalent dual modes of action (FIG. 31-35): 1) mitochondria recovery by mitophagy and mitochondria biogenesis, 2) reduced accumulation of pathological α-Synuclein.

Auto-ubiquitination was assessed on iCP-Parkin and iCP-mParkin to determine their enzymatic activity. To analyze biochemical activity of iCP-mParkin as an E3 ubiquitin ligase, auto-ubiquitination assay was carried out in test tube. iCP-Parkin ubiquitinated itself in vitro, as demonstrated with antibodies against ubiquitin (FK2) (FIG. 29). As consistent with the results of iCP-Parkin, iCP-mParkin was also able to auto-ubiquitinate either in the presence or absence of ATP. These results imply that even though iCP-mParkin did not contain UBL domain, iCP-mParkin an active E3 ligase while CI-iCP-Parkin is catalytically inactive with no E3 ligase ubiquitin activity.

Previously, we presented that iCP-Parkin could protect neuronal cells from neurotoxins [1-methyl-4-phenylpyridinium (MPP⁺) and 6-hydroxydopamine (6-OHDA)] in a dose-dependent manner. The cytoprotective effect of iCP-mParkin was confirmed by using 6-OHDA in SH-SY5Y cells (FIG. 30). The result implied that iCP-mParkin significantly protected from cytotoxicity.

Auto-ubiquitination activity of iCP-Parkin as a E3 ubiquitin ligase. Auto-ubiquitination was assessed on iCP-Parkin and iCP-mParkin to determine their enzymatic activity. To analyze biochemical activity of iCP-Parkin and iCP-mParkin as an E3 ubiquitin ligase, Parkin E3 ligase activity in test tube was measured using an auto-ubiquitination assay (Boston Biochem) conducted according to the manufacturers' instructions. Briefly, 1 μg of purified Parkin proteins were reacted with 0.1 μM E1, 1 μM E2, 50 μM Ubiquitin and 10 μM Mg-ATP for 1 hr at 37° C., followed by western blot with anti-Ubiquitin antibody (1:1,000, Enzo Life Science).

Cytoprotective effect of iCP-mParkin. The cytoprotective effect of iCP-Parkin was confirmed by using neurotoxin, 6-hydroxydopamine (6-OHDA). Human brain tumor (SH-SY5Y) cells (Korea Cell Line Bank) are cultured, plated, and SH-SY5Y cells at 70% confluence were pre-treated with 30 μM 6-OHDA and 10 μM Parkin recombinant proteins for 24 h at 37° C., and assessed for cytoprotective assay by CellTiter-Glo® 2.0 Assay (Promega). The CellTiter-Glo® 2.0 Assay provides a homogeneous method to determine the number of viable cells in culture by quantitating the amount of ATP present, which indicates the presence of metabolically active cells. Cell viability was evaluated by CellTiter-Glo cell viability assay and quantified using luminescence plate reader (Synergy H1, Biotek Instruments).

(3) Mode of Action (MoA 1 & 2): iCP-mParkin Rescues Neurons from Accumulation of Damaged Mitochondria (1) and Pathological α-Synuclein (2)

Parkin is involved in mitophagy, one of autophagy process to remove damaged mitochondria. Mitochondrial damage induced by treatment of chemicals such as CCCP, results in a series of mitophagy process by accumulation of PINK1 and subsequent Parkin activation on the mitochondria. Therefore, we hypothesized that iCP-mParkin treatment might accelerate mitophagy under mitochondria-damaged condition. The promotion of mitophagy flux in this study were correlated with elevated localization of mitochondria into lysosome or increased mitophagy under toxin-treated condition. Mitophagy flux was analyzed by measuring the level of LC3B-II, an autophagy marker located on the autophagosome membrane.

CCCP treatment gradually increased the levels of LC3B-II/LC3B-I ratio. However, iCP-mParkin further enhanced LC3B-II/LC3B-I ratio in CCCP treated cells over time, consistent with promotion of mitophagy after iCP-mParkin treatment (FIG. 31). iCP-Parkin also increased the expression of genes involved in mitochondrial biogenesis: peroxisome proliferator-activated receptor gamma coactivator 1α (PGC-1 α), transcription factor A, mitochondrial (TFAM), and nuclear respiratory factor 1 and 2. Furthermore, iCP-Parkin also recovered the cellular reactive oxygen species (ROS) levels decreased by CCCP. Therefore, these data indicate that iCP-mParkin promotes mitophagy & mitochondria biogenesis (FIG. 31-33) to replace damaged mitochondria.

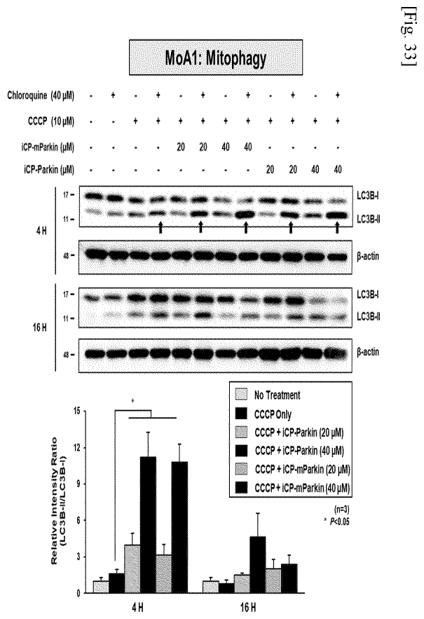

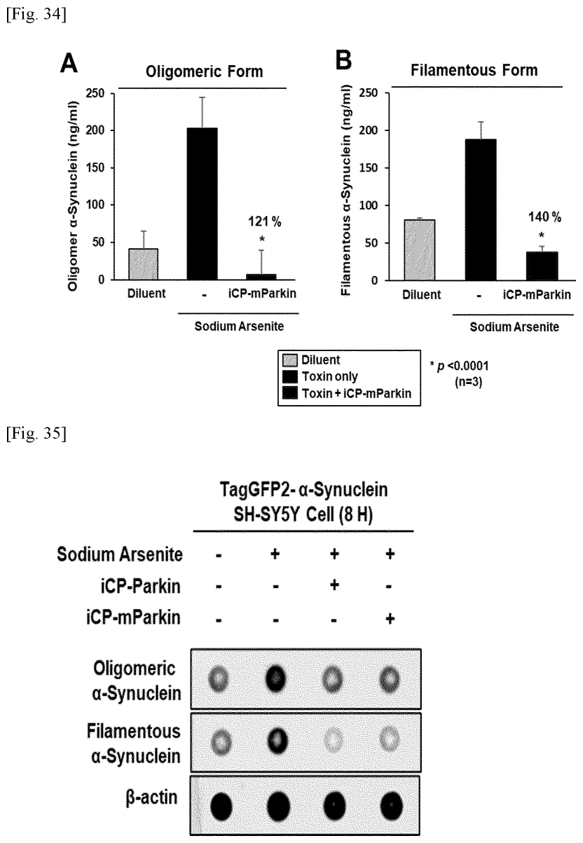

Sporadic Parkinson's disease is associated with structures known as Lewy bodies that contain pathological (oligomeric, filamentous, and phosphorylated) forms of α-Synuclein protein as well as Synphilin-1 (α-Synuclein-interacting protein) and Pael-R (one of accumulated proteins in Lewy body). Synphilin-1 and Pael-R are known Parkin substrates; whereas, despite conflicting reports α-Synuclein does not appear to be a Parkin substrate, except when the protein is glycosylated. By using SH-SY5Y cells engineered to express α-Synuclein tagged with a green fluorescent protein (TagGFP2-α-Synuclein), we examined whether iCP-mParkin influenced the levels of α-Synuclein deposits induced by sodium arsenite (NaAsO2). We confirmed that sodium arsenite increased the levels of oligomeric/filamentous α-Synuclein and iCP-mParkin reduced the levels of aggregated the levels of oligomeric/filamentous α-Synuclein (FIGS. 34 and 35).

iCP-Parkin-mediated mitophagy under mitochondria-damaged condition. Parkin is involved in mitophagy, one of autophagy process to remove damaged mitochondria. Mitochondrial damage induced by treatment of chemicals such as CCCP, results in a series of mitophagy process by accumulation of PINK1 and subsequent Parkin activation on the mitochondria. In order to examine the promotion of mitophagy flux, the level of LC3B-II, an autophagy marker located on the autophagosome membrane was analyzed by treatment of CCCP (10 μM), chloroquine (40 μM), and iCP-mParkin (40 μM). After incubation, cell lysates were subjected to western blot analysis to measure the levels of LC3B-II/LC3B-I ratio. To visualize mitochondria and mitochondria undergoing mitophagy, cells were stained with 1 mM Lyso Dye (Dojindo Molecular Technologies) and 100 nM Mtphagy Dye (Dojindo Molecular Technologies), respectively.

iCP-Parkin-mediated mitochondrial Biogenesis under mitochondria-damaged condition. Total RNA was extracted with Ribospin (GeneAll), and cDNA was synthesized from total RNA (2 μg) using a Hyperscript™ First Strand Synthesis Kit (GeneAll). Aliquots of cDNA were used as templates for the real-time qRT-PCR procedure. Relative quantities of mRNA expression were analyzed using real-time PCR (CFX96 Touch™ Real-Time PCR Detection System, (Bio-Rad). The SsoAdvanced Universal Reagents (Bio-Rad) was used according to the manufacturer's instructions. The primer sequences are described as follows: hRPLP0 (h36B4) forward (5′-TGCATCAGTACCCCATTCTATCA-3′) with reverse (5′-AAGGTGTAATCCGTCTCCACAGA-3′); hPGC1α (PPARGC1A) forward (5′-CTCAAAGACCCCAAAGGATG-3′) with reverse (5′-TGGAATATGGTGATCGGGAA-3′); hTFAM forward (5′-AGCTCAGAACCCAGATGC-3′) with reverse (5′-CCACTCCGCCCTATAAGC-3′); hNRF1 forward (5′-GGCTACCATAGAAGCACATG-3′) with reverse (5′-GAAGAAGGCGAGTCTTCATC-3′); hNRF2 (GABPA) forward (5′-ACATCAATGAACCAATAGGC-3′) with reverse (5′-CCTTGGTCAAATAAACTTCG-3′). Parkin is involved in mitophagy, one of autophagy process to remove damaged mitochondria. Mitochondrial damage induced by treatment of chemicals.

Measurement of relative oxidative stress (ROS). The SH-SY5Y cell were added into 96-well plate (4×104 cells/well). After 48 hours, washing was performed with ROS buffer. 25 μM of DCFDA (Cellular ROS Assay Kit, Abcam, ab113851) was treated in ROS buffer and reacted for 45 minutes at 100 μl in each well. Then, after washing with ROS buffer, 20-80 μM of CCCP and 20 μM of iCP-mParkin were diluted in ROS buffer and treated in wells. After 6 hours incubation, the DCFDA fluorescence intensity representing the ROS level was detected using microplate system with the excitation and emission wavelengths set as 488 and 535, respectively.

In vitro studies Assessment of Degradation of α-Synuclein aggregates in TagGFP2-α-Synuclein-expressing SH-SY5Y neuronal cells. A novel green fluorescent SH-SY5Y cell line has been developed through stable transfection with TagGPF2-α-synuclein. TagGFP2-α-synuclein cell line is stably-transfected and it is ready to use in cell-based assay applications. This stably transfected cell line provides consistent levels of α-synuclein expression. This cell line is intended to be used as an “in vitro” model for Parkinson's disease research studies. Arsenite is a ubiquitous environmental potent toxic metal. Arsenic induces a loss of mitochondrial membrane potential and induces the generation of reactive oxygen species (ROS) and lipid peroxidation. TagGFP2-α-Synuclein-expressing SH-SY5 cells were treated with iCP-mParkin (20 μM) and sodium arsenite (20 μM). After incubation, cell lysates were subjected to ELISA analysis with Anti-Alpha-synuclein aggregate antibody and human alpha-synuclein Gly111-Tyr125 antibody.

(4) In Vivo Toxicity of iCP-Parkin and iCP-mParkin

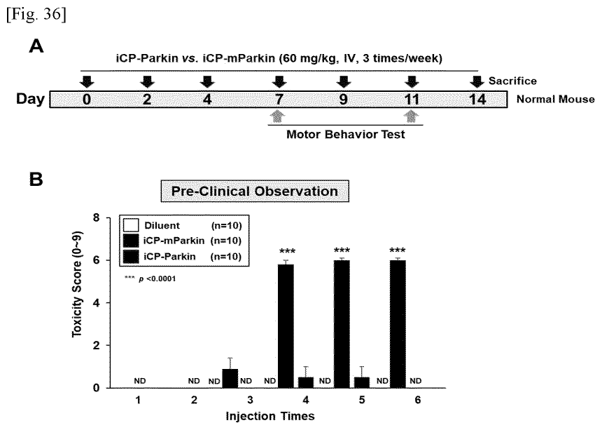

To investigate whether the new developed iCP-Parkin has in vivo toxicity, general toxicity scoring was measured. For these 2 groups of mice were treated with 60 mg/kg of iCP-Parkin and iCP-mParkin. Each group were injected intravenously 3 times per week for 2 weeks. After injection, body weight, fur condition and behavior of mice were analyzed. The group of mice injected with iCP-Parkin showed toxicity score compared to iCP-mParkin (FIG. 36).

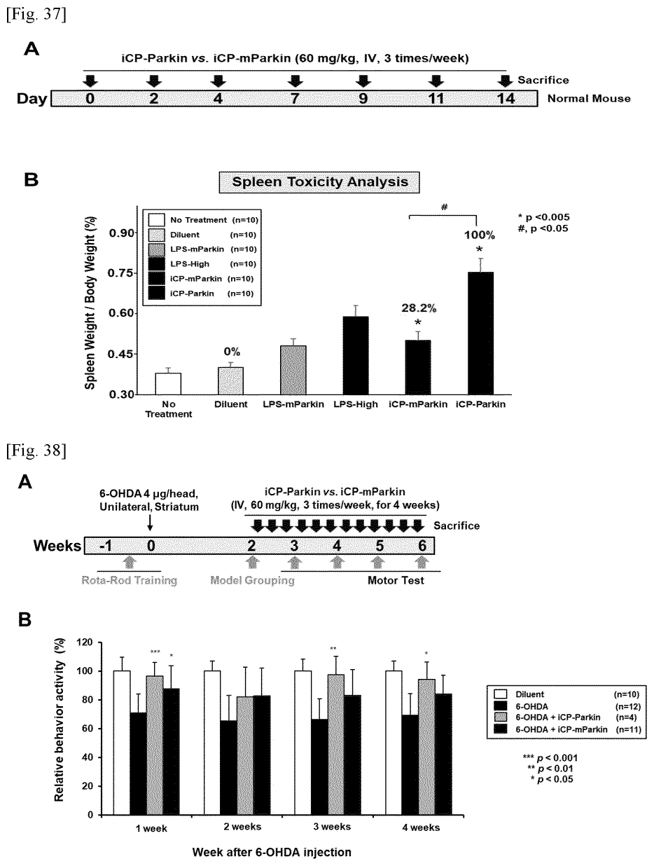

To investigate whether the newly developed iCP-mParkin also has in vivo toxicity, spleen toxicity analysis was measured. For these 2 groups of mice were treated with 60 mg/kg of iCP-Parkin and iCP-mParkin. Each group were injected intravenously 3 times per week for 2 weeks. After injection, the ratio between spleen weight and body weight of mice was analyzed. The group of mice injected with iCP-Parkin showed high spleen toxicity score compared to iCP-mParkin (FIG. 37). These results suggested that iCP-mParkin has no in vivo toxicity.

In vivo toxicity of iCP-Parkin and iCP-mParkin. iCP-Parkin and iCP-mParkin (60 mg/kg) was intravenously injected 3 times per week for 2 weeks. General toxicity scoring (including body weight, fur condition and behavior of mice) and the ratio between spleen weight and body weight were analyzed.

(5) In Vivo Efficacy of iCP-Parkin and iCP-mParkin

The neuroprotective activity of iCP-Parkin and iCP-mParkin were tested in a 6-hydroxydopamine (6-OHDA)-induced mouse model To determine the therapeutic efficacy of iCP-Parkin and iCP-mParkin administration, 4-week injection protocol was carried out in 6-OHDA-induced PD mouse model (FIG. 38). iCP-Parkin and iCP-mParkin was intravenously injected 3 times per week for 4 weeks. The rota-rod test showed the similar recovery of motor dysfunction from mice treated with iCP-Parkin and iCP-mParkin.

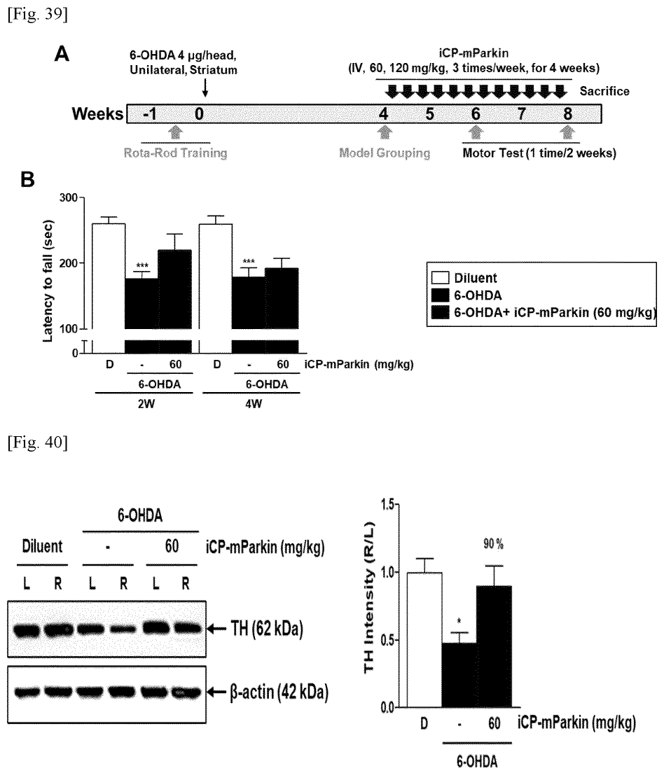

To determine the therapeutic efficacy of iCP-mParkin administration, 4-week injection protocol was carried out in 6-OHDA-induced PD mouse model. iCP-mParkin was intravenously injected 3 times per week for 4 weeks. The rota-rod test showed the treatment with iCP-mParkin improved motor dysfunction (FIGS. 39 and 40). Also, the treatment with iCP-mParkin recovered the level of TH expression (90%) in PD mouse model.

6-OHDA-induced PD mouse model. iCP-Parkin was tested in several animal models for the ability to prevent and/or restore PD-related motor symptoms. In each model, the onset of motor symptoms was verified by an apomorphine rotation test prior to starting iCP-mParkin treatments. Animals were injected subcutaneously with 0.1 mg/kg of apomorphine (freshly dissolved in 0.1% ascorbic acid solution and kept on ice in the dark before use) and judged to be symptomatic if side-biased rotation of lesioned mice turns faster than a rate of ˜60 turns over 20 min. Parkin proteins were administered intravenously (i.v.) at the times and doses as described in the text, and changes in motor function were monitored as described below. C57BL/6 mice (13 weeks male) were anesthetized by a 3:7 mixture of Alpaxan:Rompun (Bayer); positioned onto a stereotaxic apparatus and injected with 4 μg 6-OHDA (Sigma-Aldrich) dissolved in 0.8 μL of 0.02% ascorbic acid (Sigma-Aldrich) at a rate of 0.2 μL/min into the striatum at the following coordinates (relative to bregma): anterior-posterior (AP)=+0.6 mm, medial-lateral (ML)=−2.2 mm and dorsal-ventral (DV)=−3.2 mm (from the dura) with a flat skull position. Control mice were injected with 0.02% ascorbic acid solution alone.

Rota-rod test. Mice were pretrained on a Rota-rod apparatus at 15 rpm for 300 or 720 seconds to achieve stable performance. The test was conducted with a gradually accelerated speed from 4 to 40 rpm over a period of 300 seconds and recorded the time each mouse was able to stay on the rod. Each animal was tested 3 times.

Western blot analysis for tyrosine hydroxylase in animal studies. To analyze tyrosine hydroxylase in brain samples, the isolated brains were homogenized in pro-prep lysis buffer (iNtRON Biotechnology) containing a protease inhibitor (Thermo Fisher Scientific). The quantified cell lysates were separated by 10% SDS-PAGE and transferred to nitrocellulose membranes (Bio-Rad). Membranes were incubated with primary antibodies against TH (1:2,000; Millipore, AB152) and β-actin (1:100,000; Sigma-Aldrich, A3854) followed by secondary antibodies. After visualization using Supersignal West Dura (Thermo Fisher Scientific), immunoblots were quantified with ImageJ software.

2-2. Additional Indication (Alzheimer's Disease, AD): Efficacy of iCP-mParkin in AD Model

(1) Efficacy of Cognitive Function Improvement after Administration of iCP-mParkin in Fibril Amyloid-Beta (fAβ)-Induced AD Model for 2 Weeks

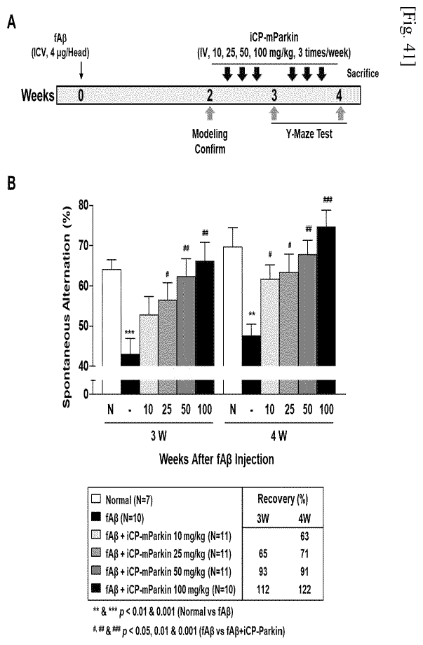

AD mouse model was induced by injecting 4 μg of fibril amyloid-beta (fAβ) into the brain through stereotaxic surgery. Two weeks after surgery, whether AD mouse model was established was verified through the Y-maze test, a cognitive function (spontaneous alternation) test. After animals were randomly grouped, iCP-mParkin was administered intravenous (IV) injection for 3 times a week for total 2 weeks in a dose-dependent manner. It was verified whether cognitive function was improved by iCP-mParkin administration through Y-maze test at 3 and 4 weeks (FIG. 41A). As a result, when iCP-mParkin was administered at 4 weeks, cognitive function was improved by 122% at 100 mg/kg of iCP-mParkin (FIG. 41B).

Animals. The study was carried out with age-matched, C57BL/6 male mice (7-8 weeks old), which were obtained from Daehan Bio Link (Eumseong-gun, Korea). The animals were kept in groups of 5 in the institutional animal room in which the temperature (set point 23±2° C.), relative air humidity (set point 50%) and light conditions (lights on/off at 8:00-20:00 H) were tightly controlled. Tap water and standard laboratory chow were provided ad libitum throughout the study.

Fibril amyloid-beta (fAβ)-induced AD mouse model. C57BL/6 male mice were anesthetized by a 7:3 mixture of Alfaxan: Rompun, positioned onto a stereotaxic apparatus, and injected with 4 μg of fAβ (rPeptide, A1170) into the brain through stereotaxic surgery at the following coordinates (relative to the bregma): anterior-posterior (AP): −2 mm, medial-lateral (ML): 0 mm, and dorsal-ventral (DV): −3 mm (from the dura) with a flat skull position. Control mice were injected with 0.01% ascorbic acid solution alone.

Behavior test (Y-maze). AD mouse model was established was verified through the Y-maze test, a cognitive function (spontaneous alternation) test. The Y-maze apparatus (arm length: 35 cm, wall height: 9 cm) consisted of three equal length arms made of white polyvinylchloride (PVC) joined in the middle to form a “Y” shape. This ethologically relevant test is based on the rodents' innate curiosity to explore novel areas and presents no negative or positive reinforcers and very little stress for the mice. Activate the video recording system immediately after placement of the mouse into the Y-maze. Press play and record the spontaneous behavior for each mouse for a period of 8 min. Once a session is complete, gently place the mouse back into its home cage and return the cage to the rack. Clean the maze thoroughly between each session with an unscented bleach germicidal wipe, 70% EtOH. Measurement of spontaneous alternation occurs when a mouse enters a different arm of the maze in each of 3 consecutive arm entries. The percentage of alternation was calculated as the ratio of actual to maximum number of alternations.

(2) Efficacy of Cognitive Function Improvement after Administration of iCP-mParkin in fAβ-Induced AD Model for 4 Weeks

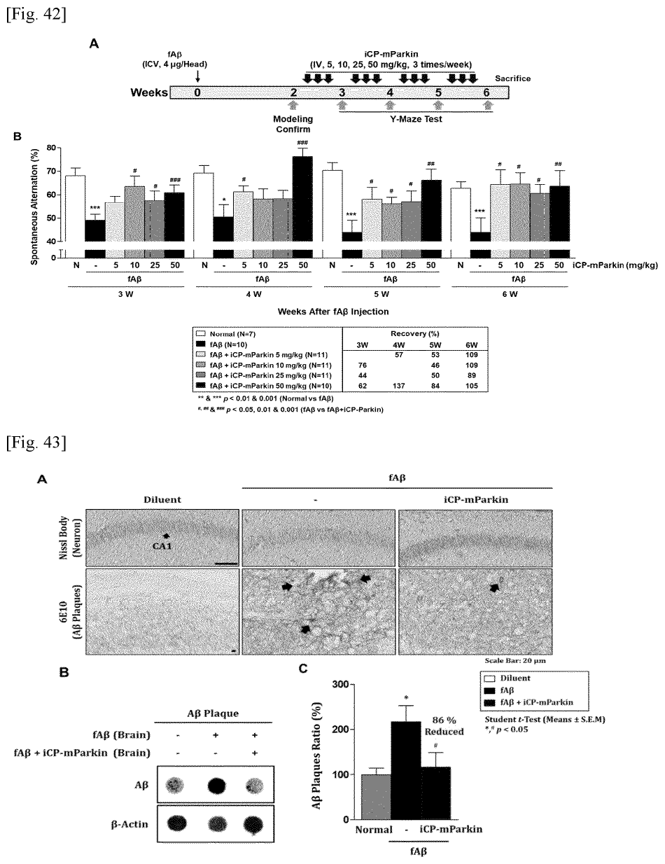

AD mouse model was induced by injecting 4 μg of fibril amyloid-beta (fAβ) into the brain through stereotaxic surgery. Two weeks after surgery, whether AD mouse model was established was verified through the Y-maze test. After animals were randomly grouped, iCP-Parkin was administered IV injection for 3 times a week for total 4 weeks in a dose-dependent manner. It was verified whether cognitive function was improved by iCP-mParkin administration through Y-maze test at 3, 4, 5 and 6 weeks (FIG. 42A). When iCP-Parkin was administered to AD model, cognitive function was improved in a time- and dose-dependent manner. Also, when iCP-mParkin was administered at 6 weeks, cognitive function was improved by 105% at 50 mg/kg of iCP-mParkin (FIG. 42B).

Animals. The study was carried out with age-matched, C57BL/6 male mice (7-8 weeks old), which were obtained from Daehan Bio Link (Eumseong-gun, Korea). The animals were kept in groups of 5 in the institutional animal room in which the temperature (set point 23±2° C.), relative air humidity (set point 50%) and light conditions (lights on/off at 8:00-20:00 H) were tightly controlled. Tap water and standard laboratory chow were provided ad libitum throughout the study.

Fibril amyloid-beta (fAβ-induced AD mouse model. C57BL/6 male mice were anesthetized by a 7:3 mixture of Alfaxan: Rompun, positioned onto a stereotaxic apparatus, and injected with 4 μg of fAβ (rPeptide, A1170) into the brain through stereotaxic surgery at the following coordinates (relative to the bregma): anterior-posterior (AP): −2 mm, medial-lateral (ML): 0 mm, and dorsal-ventral (DV): −3 mm (from the dura) with a flat skull position. Control mice were injected with 0.01% ascorbic acid solution alone.

Behavior test (Y-maze). AD mouse model was established was verified through the Y-maze test, a cognitive function (spontaneous alternation) test. The Y-maze apparatus (arm length: 35 cm, wall height: 9 cm) consisted of three equal length arms made of white polyvinylchloride (PVC) joined in the middle to form a “Y” shape. This ethologically relevant test is based on the rodents' innate curiosity to explore novel areas and presents no negative or positive reinforcers and very little stress for the mice. Activate the video recording system immediately after placement of the mouse into the Y-maze. Press play and record the spontaneous behavior for each mouse for a period of 8 min. Once a session is complete, gently place the mouse back into its home cage and return the cage to the rack. Clean the maze thoroughly between each session with an unscented bleach germicidal wipe, 70% EtOH. Measurement of spontaneous alternation occurs when a mouse enters a different arm of the maze in each of 3 consecutive arm entries. The percentage of alternation was calculated as the ratio of actual to maximum number of alternations.

(3) Elimination of Pathological Aβ Plaque from the Brain of AD Model

After all mice behavioral experiments were completed, the brain was removed. Cresyl violet staining showed that neurons were decreased in the hippocampus of the brain in fAβ-induced AD mice, and that iCP-mParkin had a neuroprotective effect. Also, Immunohistochemistry staining showed that Aβ plaques were removed from the hippocampus by iCP-mParkin (FIG. 43A). Dot blot analysis showed that Aβ plaques were reduced by 86% in the AD brain by iCP-mParkin (FIGS. 43 B & C).

Immunohistochemistry. Mice were deeply anesthetized with a Alfaxan: Rompun mixture and were perfused with saline and 4% paraformaldehyde (PFA; BIOSESANG) for 15 to 20 min. Brains were quickly fixed with 4% PFA for 2 hours at 4° C., incubated with 30% sucrose (DAEJUNG) at 4° C. for 48 hours, and embedded with optimal cutting temperature (OCT) compound (Leica Biosystems), and cryosections (20 μm thickness) were cut. Endogenous peroxidase activity was blocked by incubating the sections with 0.3% H₂O₂ (DAEJUNG) in PBS for 30 min. After washing in PBS, sections were incubated with blocking solution (5% normal goat serum in PBS; Vector Laboratories, S-1000) for 60 min. Sections were incubated with mouse monoclonal 6E10 antibody (1:250; Biolegend, SIG-39320) at 4° C. for 18 hours, followed by biotinylated goat anti-mouse immunoglobulin G (IgG; 1:200; Vector Laboratories, BA9200) for 1 hour at room temperature; sections were subsequently treated with avidin-biotinylated peroxidase complex using an ABC Kit (Vector Laboratories, PK-6100) for 60 min at room temperature. The sections were then treated with 3,30-diaminobenzidine (DAB peroxidase substrate kit, Vector Laboratories) as a chromogen. Permanently mounted slides were observed and photographed using a microscope equipped with a digital imaging system (DSRi2, Nikon). Attach the brain tissue section to the slide. Brain sections were washed with PBS or 2 hours to remove the storage buffer and allowed to dry completely. The slides were subsequently hydrated in DW for 5 min. Before being stained with 0.1% Cresyl violet Acetate (Abcam, ab246817) for 5 min. Sections were rinsed in 3 changes of DW and placed in 70→80→90→100% ethanol for 1 min. Sections were allowed to dry and then cleared by dipping in xylene before being cover-slipped and viewed using a digital imaging system (DSRi2, Nikon).

Dot blot analysis. The dot blot analysis is like western blot analysis and the experimental technique. Insoluble cell fractions were prepared as described elsewhere. For dot blot analysis, cell lysates (10 μg of protein) were bound to nitrocellulose membranes with a Bio-Dot microfiltration apparatus (Bio-Rad) by gravity filtration. This passive filtration is necessary for quantitative antigen binding.

(4) iCP-mParkin Reduces the Level of Relative Oxidative Stress (ROS) Level Caused by Aβ in HT22 Cells

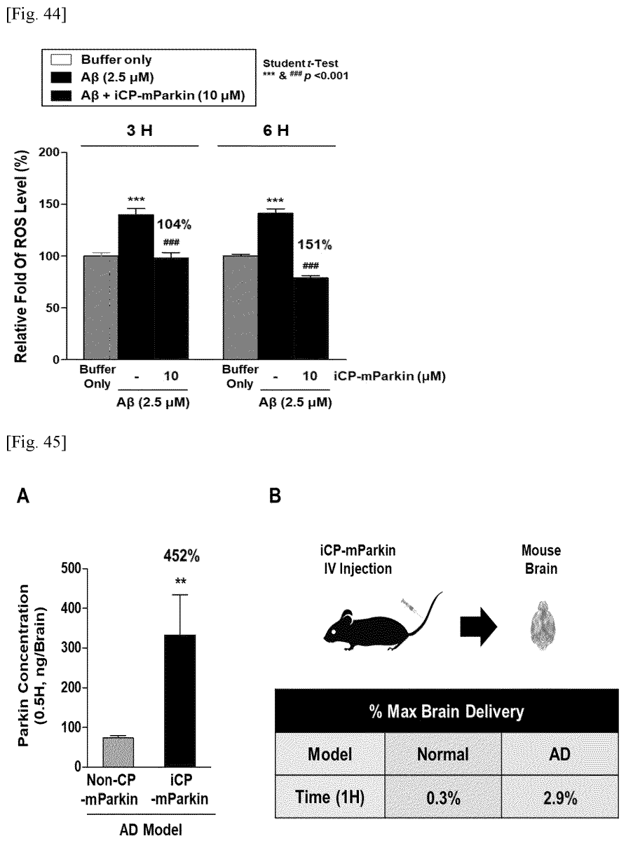

Relative oxidative stress (ROS) levels were reduced by 104% and 151% at 3 and 6 hours with iCP-mParkin (10 μM) treatment in Aβ (2.5 μM) treated HT22 cells (FIG. 44).

Measurement of relative oxidative stress (ROS). The HT22 cell (mouse hippocampal neuronal cell) were added into 96-well plate (1×10⁴ cells/well). After 24 hours, washing was performed with ROS buffer. 25 μM of DCFDA (Cellular ROS Assay Kit, Abcam, ab113851) was treated in ROS buffer and reacted for 45 minutes at 100 μl in each well. Then, after washing with ROS buffer, 2.5 μM of Aβ and 10 μM of iCP-mParkin were diluted in ROS buffer and treated in wells. After 3, 6 hours incubation, the DCFDA fluorescence intensity representing the ROS level was detected using microplate system with the excitation and emission wavelengths set as 488 and 535, respectively.

(5) Concentration of Parkin & Delivery of iCP-mParkin in the Brain of AD Model

With the TSDT platform, the concentration of parkin present in the brain is 452% higher than without the TSDT platform (FIG. 45A). In AD model, iCP-mParkin max delivered 2.9% more brain to brain compared to normal (FIG. 45B).

Measurement of iCP-mParkin By LC-MS/MS. Signature peptides from the SDB region of iCP-mParkin were detected by LC-MS/MS analysis of brain tissues [under contract with Envigo (Huntingdon, UK)]. Briefly, brain lysates were digested using two different digestion kits, SMART Digest (Thermo Fisher Scientific) and ProteinWorks (Waters), and peptides were separated on an ACE UltraCore Super C18 column (Advanced Chromatography Technologies) with an acetonitrile gradient and analyzed on a Sciex API 6500+ mass spectrometer (SCIEX).

1. An iCP (improved cell-permeable)—mParkin recombinant protein, which comprises:
i) a modified Parkin protein; and
ii) an advanced macromolecule transduction domain (aMTD);
wherein the modified Parkin protein has an amino acid sequence of SEQ ID NO:1, and the aMTD has an amino acid sequence selected from the group consisting of SEQ ID NOs: 2-241.

2. The iCP-mParkin recombinant protein according to claim 1, further comprising one or more solubilization domains (SDs).

3. The iCP-mParkin recombinant protein according to claim 2, wherein the recombinant protein is represented by any one of the following structural formulae:
A-B,
B-A,
A-B-C,
A-C-B,
B-A-C,
B-C-A,
C-A-B,
C-B-A, and
A-C-B-C
wherein
A is an advanced macromolecule transduction domain (aMTD),
B is a modified Parkin protein, and
C is a solubilization domain (SD).

4. The iCP-mParkin recombinant protein according to claim 2, wherein the recombinant protein has an amino acid sequence of SEQ ID NO: 243.

5. The iCP-mParkin recombinant protein according to claim 2, wherein the SDs have an amino acid sequence of SEQ ID NO: 242.

6. The iCP-mParkin recombinant protein according to claim 1, wherein the recombinant protein is used for treating neurodegenerative disease wherein the neurodegenerative disease is parkinson's disease, alzheimer's disease, or huntington's disease.

7. A polynucleotide sequence encoding the iCP-mParkin recombinant protein of claim 1.

8. A recombinant expression vector comprising the polynucleotide sequence of claim 7.

9. A transformant transformed with the recombinant expression vector of claim 8.

10. A composition comprising the iCP-mParkin recombinant protein of claim 1 as an active ingredient.

11. A pharmaceutical composition for treating neurodegenerative disease comprising the iCP-mParkin recombinant protein of claim 1 as an active ingredient; and a pharmaceutically acceptable carrier, wherein the neurodegenerative disease is parkinson's disease, alzheimer's disease, or huntington's disease.

12. (canceled)

13. A medicament comprising the iCP-mParkin recombinant protein of claim 1.

14. Use of the iCP-mParkin recombinant protein of claim 1 for the preparation of a medicament for treating neurodegenerative disease, wherein the neurodegenerative disease comprises parkinson's disease, alzheimer's disease, and huntington's disease.

15. A method of treating neurodegenerative disease in a subject comprising:
administering to the subject a therapeutically effective amount of the iCP-mParkin recombinant protein of claim 1.

16. A method of preparing the iCP-mParkin recombinant protein of claim 1, which comprises:
preparing the recombinant expression vector that encodes the recombinant protein;
preparing a transformat using the recombinant expression vector;
culturing the transformat; and
obtaining the recombinant protein expressed by the culturing.

17. The method of claim 16, which further comprises recovering the recombinant protein by
washing of an inclusion body;
performing the first ion exchange chromatography; and
performing the second ion exchange chromatography.

18. The method of claim 17, wherein the washing comprises one step washing using pH 8 washing buffer.

19. The method of claim 17, wherein
the first ion exchange chromatography is a cation exchange chromatography, and
the second ion exchange chromatography is an anion exchange chromatography.

20. The method of claim 19, wherein the second ion exchange chromatography comprises:
washing in an 8.0 mS/Cm conductivity condition, and
elution in a 9.0 mS/Cm conductivity condition.

21. The method of claim 16, wherein the culturing comprises a fed-batch fermentation.
